(19) United States
(12) Patent Application Publication
(10) Pub. No.: US 2023/0293521 A1
(43) Pub. Date: Sep. 21, 2023

(54) METHODS FOR SCREENING NOVEL CORONAVIRUS ANTIVIRALS AND METHODS OF USING ANTIVIRALS FOR THE TREATMENT OF CORONAVIRUS INFECTIONS

(71) Applicant: GEORGIA STATE UNIVERSITY RESEARCH FOUNDATION, INC., Atlanta, GA (US)

(72) Inventors: Christopher Francis Basler, Atlanta, GA (US); Jesus Silvas, Senoia, GA (US); Alexander Jureka, Decatur, GA (US)

(21) Appl. No.: 18/015,880

(22) PCT Filed: Jul. 14, 2021
(86) PCT No.: PCT/US2021/041584

§ 371 Date: Jan. 12, 2023

Related U.S. Application Data
(60) Provisional application No. 63/051,602, filed on Jul. 14, 2020.

(51) Int. Cl. A61K 31/506; C12Q 1/18; A61K 31/5377; A61K 31/4545; A61K 31/337; A61K 31/655; A61K 31/196; A61K 31/20; A61P 31/14

(52) U.S. Cl. CPC A61K 31/506; A61K 31/196; A61K 31/20; A61K 31/337; A61K 31/4545; A61K 31/5377; A61K 31/655; A61P 31/14; C12Q 1/18

(57) ABSTRACT

Disclosed are methods for screening for therapeutic agents that can inhibit a coronavirus infection and methods of using agents identified by said assays to treat a coronavirus infection.

This invention was made with government support under Grant Number AI125453 awarded by National Institute of Health. The government has certain rights in the invention.

### BACKGROUND

SARS-CoV-2 is a beta-coronavirus, enveloped positive-sense, RNA virus, causing a pandemic for which there is an urgent need to understand virus replication requirements and identify therapeutic strategies. Therapeutics targeting replication of SARS coronavirus 2 (SARS-CoV-2) are urgently needed to treat COVID-19 patients. Repurposing drugs developed for other purposes can provide a shortcut to therapeutic development. What are needed are new therapeutics and methods for identifying therapeutic agents that can be used to treat coronavirus infections.

### SUMMARY

Disclosed are methods and compositions related to treating and/or preventing coronavirus, tombusvirus, hepatitis C virus, and/or rotavirus infections and methods of screening for therapeutic agents that can be used in said methods.

Disclosed herein are methods of treating, reducing, inhibiting, decreasing, ameliorating and/or preventing a coronaviral, tombusviral, hepatitis C virus, and/or rotaviral infection in a subject comprising administering to the subject an inhibitor of PI3K (such as, for example, a VPS34 inhibitor including, but not limited to VPS34 IN1, VPS34 IN2 (commonly referred to as PIK-III), SB02024, compound 19, or SAR405 HY12481), an inhibitor of lipases and/or fatty acid synthase (such as, for example, Orlistat or C75), an inhibitor of fatty acyl-CoA-synthetases (such as, for example, Triacsin C), an inhibitor of diacylglycerol acyltransferase 1 (DGAT1) (such as, for example, A922500), a palmitoyl acyltransferases (PAT) inhibitor (such as, for example, 2-bromopalmitate), and/or an inhibitor of fatty acyl-CoA-carboxylase (such as, for example, 5-(Tetradecyloxy)-2-furoic acid (TOFA)). In one aspect, the inhibitor can be an antibody, siRNA, small molecule, peptide, or protein.

Also disclosed herein are methods of treating, reducing, inhibiting, decreasing, ameliorating and/or preventing a coronaviral infection of any preceding aspect, wherein the coronavirus comprises avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2, or middle east respiratory syndrome (MERS) coronavirus (CoV) (MERS-CoV); wherein the tombusvirus comprises artichoke mottled crinkle virus (AMCV), carnation Italian ringspot virus (CIRV), cucumber Bulgarian virus (CBLV), cucumber necrosis virus (CuNV), cumbidium ringspot virus (CymRSV), eggplant mottled crinkle virus (EMCV), grapevine Algerian latent virus (GALV), tomato bushy stunt virus (TBSV), Petunia asteroid mosaic virus (PetAMV), Pelargonium leaf curl virus (PLCV), Moroccan pepper virus (MPV), Limonium flower distortion virus (LFDV), Havel River virus (HRV), Lato river virus (LRV). Sitke waterborne virus (SWBV), or Neckar River virus (NRV); and/or wherein the rotavirus comprises rotavirus A, rotavirus B, rotavirus C, rotavirus D, rotavirus E, rotavirus F, rotavirus G, rotavirus H, rotavirus I, or rotavirus J.

In one aspect, disclosed herein are methods of screening for a therapeutic agent for inhibiting, reducing, and/or preventing a viral infection, the method comprising contacting a monolayer of cells with the therapeutic agent (such as, for example, an inhibitor of PI3K (such as, for example, a VPS34 inhibitor including, but not limited to VPS34 IN1, VPS34 IN2, SB02024, compound 19, or SAR405 HY12481), an inhibitor of lipases and/or fatty acid synthase (such as, for example, Orlistat or C75), an inhibitor of fatty acyl-CoA-synthetases (such as, for example, Triacsin C), an inhibitor of diacylglycerol acyltransferase 1 (DGAT1) (such as, for example, A922500), a palmitoyl acyltransferases (PAT) inhibitor (such as, for example, 2-bromopalmitate), and/or an inhibitor of fatty acyl-CoA-carboxylase (such as, for example, 5-(Tetradecyloxy)-2-furoic acid (TOFA))); infecting the monolayer of cells with the virus creating an infected cell monolayer; incubating the infected cell monolayer; and measuring impedance across the monolayer; wherein a decrease in the impedance indicates viral growth, and wherein the impedance at which 50% of the monolayer cells are dead comprises the 50% inhibitor concentration (IC50), thereby indicating that the therapeutic agent treats, inhibits, reduces ameliorates, and/or prevents viral infection.

In one aspect, disclosed herein are methods of screening for a therapeutic agent for the treatment, reduction, inhibition, and/or amelioration of a viral infection, the method comprising infecting a monolayer of cells with the virus creating an infected cell monolayer; contacting the infected monolayer with the therapeutic agent (such as, for example, an inhibitor of PI3K (such as, for example, a VPS34 inhibitor including, but not limited to VPS34 IN1, VPS34 IN2, SB02024, compound 19, or SAR405 HY12481), an inhibitor of lipases and/or fatty acid synthase (such as, for example, Orlistat or C75), an inhibitor of fatty acyl-CoA-synthetases (such as, for example, Triacsin C), an inhibitor of diacylglycerol acyltransferase 1 (DGAT1) (such as, for example, A922500), a palmitoyl acyltransferases (PAT) inhibitor (such as, for example, 2-bromopalmitate), and/or an inhibitor of fatty acyl-CoA-carboxylase (such as, for example, 5-(Tetradecyloxy)-2-furoic acid (TOFA))); incubating the infected cell monolayer; and measuring impedance across the monolayer; wherein a decrease in the impedance indicates viral growth, and wherein the impedance at which 50% of the monolayer cells are dead comprises the 50% inhibitor concentration (IC50), thereby indicating that the therapeutic agent treats, inhibits, reduces ameliorates, and/or prevents viral infection.

Also disclosed herein are methods of screening a therapeutic agent of any preceding aspect, further comprising establishing an incubation time to result in 50% death of infected, but untreated cells for a given multiplicity of infection.

In one aspect, disclosed herein are methods of screening a therapeutic agent of any preceding aspect, further comprising comparing the IC50 with uninfected cell controls and/or infected and untreated cell controls. Also disclosed herein are methods of screening a therapeutic agent of any preceding aspect, further comprising comparing the IC50 with a positive control including, but not limited to an inhibitor of PI3K (such as, for example, a VPS34 inhibitor including, but not limited to VPS34 IN1, VPS34 IN2, SB02024, compound 19, or SAR405 HY12481), an inhibitor of lipases and/or fatty acid synthase (such as, for example, Orlistat or C75), an inhibitor of fatty acyl-CoA-synthetases (such as, for example, Triacsin C), an inhibitor of diacylglycerol acyltransferase 1 (DGAT1) (such as, for example, A922500), a palmitoyl acyltransferases (PAT) inhibitor (such as, for example, 2-bromopalmitate), and/or an inhibitor of fatty acyl-CoA-carboxylase (such as, for example, 5-(Tetradecyloxy)-2-furoic acid (TOFA)).

Also disclosed herein are methods of screening a therapeutic agent of any preceding aspect, wherein the therapeutic agent is added to the monolayer prior to infection (for example, wherein the therapeutic agent is added to the monolayer at least 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72 hours prior to infection) or wherein the therapeutic agent is added to the monolayer after infection (for example, wherein the therapeutic agent is added to the monolayer at least 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72 hours after infection).

In one aspect, disclosed herein are methods of screening a therapeutic agent of any preceding aspect, wherein the virus is hepatitis C virus; wherein the virus is a coronavirus selected from the group consisting of avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, delta variant, and/or P.1 variant), and middle east respiratory syndrome (MERS) coronavirus (CoV) (MERS-CoV); wherein the virus is a tombusvirus selected from the group consisting of artichoke mottled crinkle virus (AMCV), carnation Italian ringspot virus (CIRV), cucumber Bulgarian virus (CBLV), cucumber necrosis virus (CuNV), cumbidium ringspot virus (CymRSV), eggplant mottled crinkle virus (EMCV), grapevine Algerian latent virus (GALV), tomato bushy stunt virus (TBSV), Petunia asteroid mosaic virus (PetAMV), Pelargonium leaf curl virus (PLCV), Moroccan pepper virus (MPV), Limonium flower distortion virus (LFDV), Havel River virus (HRV), Lato river virus (LRV). Sitke waterborne virus (SWBV), and Neckar River virus (NRV); and/or wherein the virus is a rotavirus selected from the group consisting of rotavirus comprises rotavirus A, rotavirus B, rotavirus C, rotavirus D, rotavirus E, rotavirus F, rotavirus G, rotavirus H, rotavirus I, and rotavirus J.

In one aspect, disclosed herein are methods of inhibiting, reducing, decreasing, and/or preventing viral replication comprising contacting a virus with an inhibitor of fatty acid synthesis including but not limited to Orlistat, C75, protein palmitoylation inhibitors (such as, for example, a palmitoyl acyltransferases (PAT) inhibitor including, but not limited to 2-bromopalmitate), inhibitors of fatty acyl-CoA-synthetases (ACS)(such as, for example, Triacsin C), inhibitors of diacylglycerol acyltransferase 1 (DGAT1)(such as, for example, A922500), and/or inhibitors of fatty acyl-CoA-carboxylases (ACC)(such as, for example, TOFA).

Also disclosed herein are methods of inhibiting, reducing, decreasing, and/or preventing viral replication of any preceding aspect, wherein the virus is a coronavirus selected from the group consisting of avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, delta variant, and/or P.1 variant), and middle east respiratory syndrome (MERS) coronavirus (CoV) (MERS-CoV; hepatitis C virus; a tombusvirus selected from the group consisting of artichoke mottled crinkle virus (AMCV), carnation Italian ringspot virus (CIRV), cucumber Bulgarian virus (CBLV), cucumber necrosis virus (CuNV), cumbidium ringspot virus (CymRSV), eggplant mottled crinkle virus (EMCV), grapevine Algerian latent virus (GALV), tomato bushy stunt virus (TBSV), Petunia asteroid mosaic virus (PetAMV), Pelargonium leaf curl virus (PLCV), Moroccan pepper virus (MPV), Limonium flower distortion virus (LFDV), Havel River virus (HRV), Lato river virus (LRV). Sitke waterborne virus (SWBV), and Neckar River virus (NRV; or a rotasvirus selected from the group consisting of rotavirus A, rotavirus B, rotavirus C, rotavirus D, rotavirus E, rotavirus F, rotavirus G, rotavirus H, rotavirus I, and rotavirus J.

### BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, and 1D show standardization of an electrical resistance-based assay as a measure of SARS CoV-2 induced CPE and anti-SARS-CoV-2 activity. Vero E6 cells were seeded into a CytoView-Z 96-well plate and allowed to stabilize overnight, as measured by electrical resistance. FIG. 1A shows resistance was measured every minute over the course of 72 hours in wells that were mock infected or infected with SARS-CoV-2 in 10-fold dilutions ranging from an MOI of 10-0.0001. Solid lines indicate the mean, dotted lines indicate the standard error of three replicates. FIG. 1B shows median time-to-death calculations based on raw resistance data for each MOI. FIG. 1C shows remdesivir was titrated in 6-fold dilutions ranging from 50-0.006 µM. After infection at an MOI of 0.01, resistance was monitored for 48 hpi and FIG. 1D shows percent inhibition for Remdesivir based on the data from the 48-hour time point is presented (solid circles).

FIGS. 2A, 2B, 2C, and 2D show VPS34 exhibit anti-SARS-CoV-2 activity. Vero E6 cells were seeded into a CytoView-Z 96-well plate and allowed to stabilize overnight. Cells were pre-treated with serial half-log dilutions of VPS34-IN1 (2A-2B) or PIK-III (2C-2D) and infected with SARS-CoV-2 at an MOI of 0.01. Resistance (2A and 2C) was measured every minute over the course of 48 hours and percent inhibition (2B and 2D) was determined at the 48-hour timepoint (solid circles) as compared to the infected DMSO treated control (red). Uninfected cells are indicated in blue.

FIGS. 3A, 3B, 3C, and 3D show screening of fatty acid inhibitors for potential anti-SARS-CoV-2 activity. Vero E6 cells were seeded into a CytoView-Z 96-well plate and allowed to stabilize overnight. Cells were pre-treated with serial half-log dilutions of Orlistat (3A-3B) or Triacsin C (3C-3D) and infected with SARS CoV-2 at an MOI of 0.01. Resistance (3A and 3C) was measured every minute over the course of 48 hours and percent inhibition (3B and 3D) was determined at the 48-hour timepoint (solid circles) as compared to the infected DMSO treated control (red). Uninfected cells are indicated in blue.

FIGS. 4A and 4B show time addition studies. VeroE6 cells were seeded into a CytoView-Z 96-well plate and allowed to stabilize overnight. FIG. 4A shows a schematic of time-of-addition timeline. FIG. 4B shows that VeroE6 cells were pre-treated for one hour and compound removed (Removed) or pre-treated for one hour, 2 h.p.i. or 4 h.p.i. with compound maintained throughout infection (Maintained). TEER was measured every minute over the course of 72 hours.

FIGS. 5A, 5B, 5C, 5D, 5E, 5F, 5G, and 5H show attenuation of VPS34 kinase activity and fatty acid metabolism inhibit SARS-CoV-2 replication in human airway epithelial cell line. Calu-3 cells were plated onto a 96-well plate and allowed to reach confluency. Cells were then pre-treated with a series of 3-fold dilutions of VPS34-IN1 (5A-B), PIK-III (5C-D), Orlistat (5E-F), Triacsin C (5G-H), DMSO, or mock-treated with media alone for 1 hour, then infected with SARS-CoV-2 at an MOI of 0.01. Supernatants were collected at 48 hpi and virus was quantified by plaque assay on VeroE6 cells. The data is reported as plaque forming units per milliliter (pfu/ml) (left panels) and percent inhibition (right panels). Cell viability over 48 hours was determined in parallel and plotted with the percent inhibition data. IC50 and IC90 were calculated from the plaque assay data and are indicated on the curves. For the plaque assay data, the dotted lines labeled DMSO and LOD indicate the level of virus growth in the DMSO control and the limit of detection, respectively.

FIG. 6 shows inhibition of DGATs does not prevent SARS-CoV-2 replication. VeroE6 cells were seeded into a CytoView-Z 96-well plate and allowed to stabilize overnight. Cells were pre-treated with serial half-log dilutions of A) TC863 or B) PF06424439 and infected with SARS-CoV-2 at an MOI=0.01. Resistance was measured every minute over the course of 48 hours and percent inhibition relative to the DMSO control was determined at the 48-hour timepoint.

FIG. 7 shows inhibition of alpha PI3K does not prevent SARS-CoV-2 replication. Calu-3 cells were plated onto a 96-microplate and allowed to reach 95% confluency. Cells were then pre-treated with a range of concentrations of BYL719 and infected with SARS-CoV-2 at an MOI=0.01. Supernatants were collected at 48 h.p.i. and titered on VeroE6 cells (left panel). Cell toxicity was determined in parallel and percent inhibition extrapolated from plaque assay data (right panel).

FIGS. 8A and 8B show that SARS-CoV-2 N and nascent viral RNA co-localize with the autophagy membrane marker LC3. VeroE6 cells were infected with SARS-CoV-2. At 24 h.p.i., cells were pre-treated with actinomycin D followed by a 5-ethynyl uridine (EU) chase for 4 hours. FIG. 8A shows that cells were fixed, EU labeled viral nascent RNA was detected with click chemistry, and immunofluorescence performed using primary anti-bodies against SARS-CoV-2 N or LC3 and AlexaFluor488 or AlexaFluor647 as secondary antibodies. Nuclei were stained with Hoeschst 33342. Representative images are shown. FIG. 8B shows co-localization was analyzed with Zen Blue.

FIG. 9 shows VPS34 activity and fatty acid metabolism are required to form SARS-CoV-2 N replication centers. Calu-3 cells were pre-treated with VPS34-IN1 (5 uM), VPS34-IN2 (5 uM), Orlistat (500 uM), or Triacsin C (50uM) for 1 hour and infected with SARS-CoV-2. Cells were fixed at 24 h.p.i. and immunofluorescence performed using primary anti-bodies against SARS-CoV-2 N or dsRNA and AlexaFluor488 or AlexaFluor647 as secondary antibodies. Nuclei were stained with Hoeschst 33342. Representative images are shown.

FIGS. 10A, 10B, 10C, 10D, and 10E show mechanistic characterization of anti-SARS-CoV-2 activity. To determine which steps involved in fatty acid metabolism contribute to the observed anti-SARS-CoV-2 activity, Calu-3 cells were pre-treated with DMSO or a series of 3-fold dilutions of the indicated compounds, or mock-treated with media alone for 1 hour, then infected with SARS-CoV-2 at an MOI of 0.01. Supernatants were collected at 48 hpi and virus was quantified by focus forming assay on VeroE6 cells. Cytotoxicity assays were performed in parallel. IC50 and CC50 values were calculated for each compound (10A). To discern whether or not genomic and subgenomic RNA levels are affected by compound treatment, Calu-3 cells were pre-seeded in 24-well format, allowed to grow to confluency, and infected at an MOI of 1. 2 hours post-infection cells were treated with VPS34-IN1 (5 µM), Orlistat (500 µM), Triacsin C (5 µM), TOFA (50 µM), 2-bromopalmitate (50 µM), A922500 (30 µM), Remdesivir (1 µM), or DMSO. At 4, 10, and 24 hpi total RNA was extracted from the cell monolayers, and 24 hpi supernatants were harvested for viral titers. Virus titers were determined by plaque assay (10B). Levels of genomic RNA, subgenomic N RNA, and NSP14 RNA were quantified via qPCR (see materials and methods). Data are represented as fold change of RNA levels in infected compound treated samples versus infected DMSO treated samples (10C, 10D, and 10E). The virus titer at 24 hpi for compound treated cells (orange bars) are plotted alongside the qPCR data and represented as fold-change compared to titers from DMSO treated cells.

FIGS. 11A, 11B, and 11C show that fatty acid metabolism is essential for efficient SARS-CoV-2 replication. FASN CRISPR KO Caco2 cells and corresponding NT Caco2 cells were pre-seeded in 96-well plates, grown to confluency, and then infected at an MOI 0.01 in minimal media. Post adsorption, cells were maintained in either 2% FBS DMEM (11A) or 1% fatty-acid free (FAF)-BSA DMEM (11B). Supernatants were collected at 1, 24, 48, 72, and 96 hpi and viral titers were determined by plaque assay. Protein samples were obtained from cell monolayers and analyzed by western blot to confirm FASN knockout and look for changes in SARS-CoV-2 N levels. FASN KO and WT cells were pre-seeded and infected as previously described. Post adsorption, inoculum was removed and replaced with 2% FAF-BSA DMEM or 2% FAF-BSA DMEM containing 250 µM palmitic acid + 250 µM oleic acid (11C). Supernatants were collected at 1, 24, 48, and 72 hpi and viral titers were determined via plaque assay.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, avian, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.” For example, a negative control can be an untreated or mock treated control. A positive control, can be a control with a known positive response.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dos may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Screening

Coronavirus (CoV) replication involves multiple critical interactions with host cell membranes, including viral entry, establishment of replication centers in double membrane vesicles, and virus release. Repurposing drugs developed for other purposes can provide a shortcut to therapeutic development. To assess whether SARS-CoV-2 is susceptible to modulators of membrane metabolism/biology the sensitivity of the virus in Vero E6 and Calu-3 cells to inhibitors was assessed. To achieve this result, a 96-well format assay was created that provides real-time, hands-free monitoring of the integrity of a Vero E6 cell monolayer, thereby providing assessment of virus growth and cell viability. This assay is the first use of electrical impedance being used to measure viral growth across a monolayer. The use of electrical impedance of a viral infection provides continuous real-time, label free monitoring of the integrity of cell monolayers. Accordingly, in one aspect, disclosed herein are methods of screening for a therapeutic agent for the prevention, inhibition, and/or reduction, of a viral infection (such as, for example a coronavirus, hepatitis C virus, rotavirus, and/or tombusvirus infection), the method comprising contacting a monolayer of cells with the therapeutic agent (such as, for example, an inhibitor of PI3K (such as, for example, a VPS34 inhibitor including, but not limited to VPS34 IN1, VPS34 IN2, SB02024, compound 19, or SAR405 HY12481), an inhibitor of lipases and/or fatty acid synthase (such as, for example, Orlistat), an inhibitor of fatty acyl-CoA-synthetases (such as, for example, Triacsin C), an inhibitor of diacylglycerol acyltransferase 1 (DGAT1) (such as, for example, A922500), a palmitoyl acyltransferases (PAT) inhibitor (such as, for example, 2-bromopalmitate), and/or an inhibitor of fatty acyl-CoA-carboxylase (such as, for example, 5-(Tetradecyloxy)-2-furoic acid (TOFA))); infecting the monolayer of cells with the virus creating an infected cell monolayer; incubating the infected cell monolayer; and measuring impedance across the monolayer; wherein a decrease in the impedance indicates viral growth, and wherein the impedance at which 50% of the monolayer cells are dead comprises the 50% inhibitor concentration (IC50).

It is understood and herein contemplated that the use of the disclosed screening methods is not limited to the identification of therapeutic agents that can prevent, inhibit, and/or reduce the establishment of a viral infection, but also be used to screen for therapeutic agents that can be used to treat, decrease, inhibit, reduce, and/or ameliorate established viral infections as each round of infection requires the virus to leave a host cell and infect a new uninfected cell. Thus, an agent that inhibits, reduces, and/or prevents a new infection can also be an agent that treats, reduces, inhibits, ameliorate, and/or decreases an ongoing infection if by no other means that slowing and/or stopping viral spread. A screen for an established infection can be achieved by infecting the cells prior to the step of providing the therapeutic agent treatment. Thus, in one aspect, disclosed herein are methods of screening for a therapeutic agent for the inhibition, reduction, decrease, and/or amelioration of a viral infection (such as, for example a coronavirus, hepatitis C virus, rotavirus, and/or tombusvirus infection), the method comprising infecting the monolayer of cells with the virus creating an infected cell monolayer; contacting a monolayer of cells with the therapeutic agent (such as, for example, an inhibitor of PI3K (such as, for example, a VPS34 inhibitor including, but not limited to VPS34 IN1, VPS34 IN2, SB02024, compound 19, or SAR405 HY12481), an inhibitor of lipases and/or fatty acid synthase (such as, for example, Orlistat), an inhibitor of fatty acyl-CoA-synthetases (such as, for example, Triacsin C), an inhibitor of diacylglycerol acyltransferase 1 (DGAT1) (such as, for example, A922500), a palmitoyl acyltransferases (PAT) inhibitor (such as, for example, 2-bromopalmitate), and/or an inhibitor of fatty acyl-CoA-carboxylase (such as, for example, 5-(Tetradecyloxy)-2-furoic acid (TOFA))); incubating the infected cell monolayer in the presence of the therapeutic agent; and measuring impedance across the monolayer; wherein a decrease in the impedance indicates viral growth, and wherein the impedance at which 50% of the monolayer cells are dead comprises the 50% inhibitor concentration (IC50).

It is understood and herein contemplated that standardization of the viral infection on the cell line can be advantageous in generating a curve from which the impedance loss for treated cells is compared. Accordingly, in one aspect, disclosed herein are methods of screening a therapeutic agent of any preceding aspect, further comprising establishing an incubation time to result in 50% death of infected, but untreated cells for a given multiplicity of infection. Additionally, the disclosed screening methods can further comprise comparing the IC50 with uninfected cell controls and/or infected and untreated cell controls. Also disclosed herein are methods of screening a therapeutic agent, further comprising comparing the IC50 with a positive control including, but not limited to an inhibitor of PI3K (such as, for example, a VPS34 inhibitor including, but not limited to VPS34 IN1 (Cayman Chemicals; Catalog #17392), VPS34 IN2 (Cayman Chemicals; Catalog #17002), SB02024, compound 19, or SAR405 HY12481), an inhibitor of lipases and/or fatty acid synthase (such as, for example, Orlistat (Cayman Chemicals; Catalog #10005426) or C75(Cayman Chemicals; Catalog #10005270)), an inhibitor of fatty acyl-CoA-synthetases (such as, for example, Triacsin C (Cayman Chemicals; Catalog #10007448)), an inhibitor of diacylglycerol acyltransferase 1 (DGAT1) (such as, for example, A922500 (Cayman Chemicals; Catalog #10012708)), a palmitoyl acyltransferases (PAT) inhibitor (such as, for example, 2-bromopalmitate (Sigma-Aldrich Catalog #21604), and/or an inhibitor of fatty acyl-CoA-carboxylase (such as, for example, 5-(Tetradecyloxy)-2-furoic acid (TOFA) (Cayman Chemicals; Catalog #10005263)).

The disclosed method can be performed on any suitable cell or cell line (including primary cells and commercially available cell lines) suitable for infection with the target virus against which the therapeutic agent is applied. Examples of cells or cell lines for use in the disclosed methods include but are not limited to Vero cells, Calu-3 cells, Caco-2 cells, Madden-Darby Canine Kidney (MDCK) cells, rhesus monkey kidney cells (RhMK), primary rabbit kidney cells, MRC-5, human foreskin fibroblasts, HEp-2, and A549.

It is understood and herein contemplated that the assessment of an IC50 of a potential therapeutic agent can be applied over one or multiple dose concentrations as well as number (for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 dose administrations) and frequency of applications (such as, for example, a single administration, constant administration, or a dose every 5, 10, 15, 20, 25, 30, 35, 40, 45, 50, 60, 75, 90, 100, 120, 150, 180 minutes 4, 5, 6, 7, 8, 9, 10, 11, 12, 13, 14, 15, 16, 17, 18, 19, 20, 21, 22, 23, 24, 30, 36, 42, 48 hours, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 45, 49, 56, 58, 59, 60, 61, 62 days) of the therapeutic agent and when initial contact of the therapeutic agent and the cells occurs relative to infection (i.e., before, concurrent, simultaneous, or after infection). In one aspect, disclosed herein are methods of screening a therapeutic agent, wherein the therapeutic agent is added to the monolayer prior to infection (for example, wherein the therapeutic agent is added to the monolayer at least 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72 hours prior to infection) or wherein the therapeutic agent is added to the monolayer after infection (for example, wherein the therapeutic agent is added to the monolayer at least 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72 hours after infection).

The disclosed screening methods are designed to identify a therapeutic agent that is efficacious in treating, inhibiting, reducing, decreasing, ameliorating, and/or preventing a viral infection. It is understood and herein contemplated that the disclosed methods will work to identify a therapeutic agent irrespective of the virus being targeted but coronaviruses, tombusviruses, rotaviruses, and hepatitis C virus are particularly contemplated. Thus, in one aspect, disclosed herein are methods of screening a therapeutic agent, wherein the virus is hepatitis C virus; wherein the virus is a coronavirus selected from the group consisting of avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, delta variant, and/or P.1 variant), and middle east respiratory syndrome (MERS) coronavirus (CoV) (MERS-CoV); wherein the virus is a tombusvirus selected from the group consisting of artichoke mottled crinkle virus (AMCV), carnation Italian ringspot virus (CIRV), cucumber Bulgarian virus (CBLV), cucumber necrosis virus (CuNV), cumbidium ringspot virus (CymRSV), eggplant mottled crinkle virus (EMCV), grapevine Algerian latent virus (GALV), tomato bushy stunt virus (TBSV), Petunia asteroid mosaic virus (PetAMV), Pelargonium leaf curl virus (PLCV), Moroccan pepper virus (MPV), Limonium flower distortion virus (LFDV), Havel River virus (HRV), Lato river virus (LRV). Sitke waterborne virus (SWBV), and Neckar River virus (NRV); and/or wherein the virus is a rotavirus selected from the group consisting of rotavirus comprises rotavirus A, rotavirus B, rotavirus C, rotavirus D, rotavirus E, rotavirus F, rotavirus G, rotavirus H, rotavirus I, and rotavirus J

### C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular inhibitor of PI3K (such as, for example, a VPS34 inhibitor including, but not limited to VPS34 IN1, VPS34 IN2 (commonly referred to as PIK-III), SB02024, compound 19, or SAR405 HY12481), inhibitor of lipases and/or fatty acid synthase (such as, for example, Orlistat), an inhibitor of fatty acyl-CoA-synthetases (such as, for example, Triacsin C), an inhibitor of diacylglycerol acyltransferase 1 (DGAT1) (such as, for example, A922500), a palmitoyl acyltransferases (PAT) inhibitor (such as, for example, 2-bromopalmitate), and/or an inhibitor of fatty acyl-CoA-carboxylase (such as, for example, 5-(Tetradecyloxy)-2-furoic acid (TOFA)) is disclosed and discussed and a number of modifications that can be made to a number of molecules including the inhibitor of PI3K, lipases, fatty acid synthase, and/or fatty acyl-CoA-synthetases are discussed, specifically contemplated is each and every combination and permutation of the inhibitor of PI3K, lipases, fatty acid synthase, and/or fatty acyl-CoA-synthetases and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Agents that can be identified by the disclosed methods can be any peptide, protein, antibody, antibody fragment, chimeric antigen receptor (CAR), functionalized binding molecule (e.g., an immunotoxin), siRNA, antisense RNA or small molecule. In one aspect, the therapeutic agent can be an antibody, antibody fragment, CAR, functionalized binding molecule, siRNA, or small molecule that inhibits VPS34 including, but not limited to the following small molecule inhibitors of VPS34.

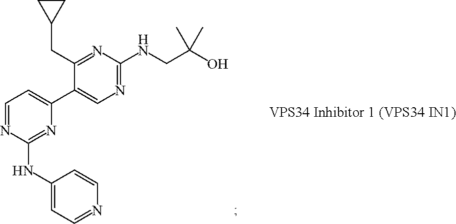

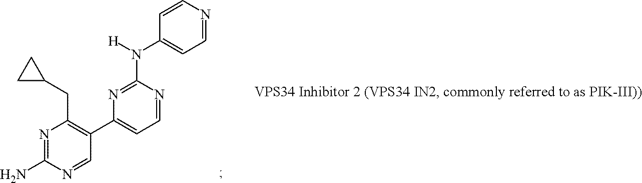

SB02024 from Sprint Biosciences AB (Noman et al. (2020) Science Advances 6(18): eaax7881 and Dyczynski et al. (2018) Cancer Letters 435: 32-43): and

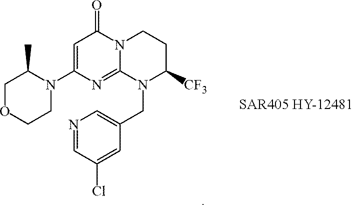

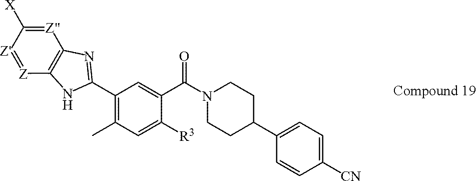

Wherein X is

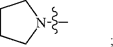

wherein R3 is a methyl (Me); wherein Z is CH; wherein Z′ is N; and wherein Z″ is CH.

### 1. Antibodies

### Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with VPS34, lipases, fatty acid synthase, and/or fatty acyl-CoA-synthetases such that the activity of the target is inhibited. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain PI3K (including, but not limited to VPS34), lipases, fatty acid synthase, and/or fatty acyl-CoA-synthetases binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M.J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### Administration of Antibodies

Administration of the antibodies can be done as disclosed herein. Nucleic acid approaches for antibody delivery also exist. The broadly neutralizing anti PI3K (including, bt not limited to VPS34), lipases, fatty acid synthase, and/or fatty acyl-CoA-synthetases antibodies and antibody fragments can also be administered to patients or subjects as a nucleic acid preparation (e.g., DNA or RNA) that encodes the antibody or antibody fragment, such that the patient’s or subject’s own cells take up the nucleic acid and produce and secrete the encoded antibody or antibody fragment. The delivery of the nucleic acid can be by any means, as disclosed herein, for example.

### 2. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K.D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### A) Pharmaceutically Acceptable Carriers

The compositions, including antibodies, can be used therapeutically in combination with a pharmaceutically acceptable carrier.

Suitable carriers and their formulations are described in Remington: The Science and Practice oƒ Pharmacy (19th ed.) ed. A.R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer’s solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer’s dextrose, dextrose and sodium chloride, lactated Ringer’s, or fixed oils. Intravenous vehicles include fluid and nutrient replenishers, electrolyte replenishers (such as those based on Ringer’s dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### B) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook oƒ Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 µg/kg to up to 100 mg/kg of body weight or more per day, depending on the factors mentioned above. It is understood and herein contemplated that the skilled artisan is aware that therapeutic dosage for one inhibitor will not necessarily be the same as another inhibitor. In one aspect, where the inhibitor is a PI3K inhibitor (such as, for example, a VPS34 inhibitor including, but not limited to VPS34 IN1, VPS34 IN2 (commonly referred to as PIK-III), SB02024, compound 19, or SAR405 HY12481) the therapeutic dosage can be between about 0.1 mg/kg to about 5 mg/kg, preferably between about 0.25 mg/kg and 2.5 mg/kg, most preferably between about 0.325 mg/kg and 2.0 mg/kg. Thus in one aspect the therapeutic dosage for a PI3K inhibitor can be about 0.1, 0.125, 0.133, 0.15, 0.167, 0.175, 0.2, 0.233, 0.25, 0.267, 0.275, 0.3, 0.325, 0.333, 0.35, 0.367, 0.375, 0.4, 0.425, 0.433, 0.45, 0.467, 0.475, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 1.91, 1.92, 1.93, 1.94, 1.95, 1.96, 1.97, 1.98, 1.99, 2.0, 2.1, 2.2, 2.3, 2.4, 2.5, 2.6, 2.7, 2.8, 2.9, 3.0, 3.1, 3.5, 3.3, 3.4, 3.5, 3.6, 3.7, 3.8, 3.9, 4.0, 4.1, 4.2, 4.3, 4.4, 4.5, 4.6, 4.7, 4.8, 4.9, or 5 mg/kg. In one aspect, the inhibitor is IN1, and the therapeutic dosage is between about 1.5 mg/kg and 2.5 mg/kg, preferably between about 1.75 mg/kg and 2.25 mg/kg, most preferably between about 1.9 mg/kg and 2.0 mg/kg. In one aspect, the inhibitor is IN1, and the therapeutic dosage is 1.97 mg/kg. In one aspect, the inhibitor is PIK-III, and the therapeutic dosage is between about 0.25 mg/kg and 0.5 mg/kg, preferably between about 0.3 mg/kg and 0.4 mg/kg, most preferably between about 0.35 mg/kg and 0.375 mg/kg. In one aspect, the inhibitor is PIK-III, and the therapeutic dosage is 0.367 mg/kg. Where the PI3K inhibitor is compound 19, the therapeutic dosage can be between about 10 mg/kg to about 100 mg/kg, preferably between about 25 mg/kg and 75 mg/kg, most preferably between about 45 mg/kg and 60 mg/kg. Thus, in one aspect the therapeutic dosage for compound 19 can be about 10, 15, 20, 25, 30, 35, 40, 45, 50, 55, 60, 65, 70, 75, 80, 85, 90, 95, or 100 mg/kg. Similarly, where the inhibitor is an inhibitor of lipases and/or fatty acid synthase (such as, for example, Orlistat or C75), the therapeutic dosage can be between about 50 mg/kg to about 150 mg/kg, preferably between about 75 mg/kg and 125 mg/kg, most preferably between about 90 mg/kg and 100 mg/kg. Thus in one aspect the therapeutic dosage for an inhibitor of lipases and/or fatty acid synthase (such as, for example, Orlistat or C75) can be about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 98.1, 98.2, 98.3, 98.4, 98.41, 98.42, 98.43, 98.44, 98.45, 98.46, 98.47, 98.48, 94.49, 98.5, 98.6, 98.7, 98.8, 98.9, 100, 101, 102, 103, 104, 105, 106, 107, 108, 109, 110, 111, 112, 113, 114, 115, 116, 117, 118, 119, 120, 121, 122, 123, 124, 125, 130, 135, 140, 145, or 150 mg/kg. In one aspect, where the inhibitor is an inhibitor of fatty acyl-CoA-synthetases (such as, for example, Triacsin C) the therapeutic dosage can be between about 0.01 mg/kg to about 0.1 mg/kg, preferably between about 0.025 mg/kg and 0..075 mg/kg, most preferably between about 0.05 mg/kg and 0.065 mg/kg. Thus in one aspect the therapeutic dosage for an inhibitor of fatty acyl-CoA-synthetases (such as, for example, Triacsin C) can be about 0.01, 0.0125, 0.015, 0.0175,, 0.02, 0.0225, 0.025, 0.0275, 0.03, 0.0325, 0.035, 0.0375, 0.04, 0.0425, 0.045, 0.0475, 0.05, 0.051, 0.052, 0.0525, 0.053, 0.054, 0.055, 0.056, 0.057, 0.058, 0.059, 0.06, 0.061, 0.062, 0.063, 0.064, 0.065, 0.0675, 0.07, 0.0725, 0.075, 0.0775, 0.08, 0.0825, 0.085, 0.0875, 0.09, 0.0925, 0.095, 0.0975, or 1.0 mg/kg.

### D. Methods of Treating a Viral Infection

Once a therapeutic agent is identified that is efficacious in the treatment, decrease, reduction, inhibition, amelioration, and /or prevention of a viral infection, it is understood and herein contemplated that the therapeutic agent can be administered to a subject at risk for acquiring a viral infection or having a viral infection. Thus, in one aspect, disclosed herein are methods of treating, reducing, inhibiting, decreasing, ameliorating and/or preventing a viral infection (such as, for example a coronaviral, rotaviral, tombusviral, and/or hepatitis C infection) in a subject comprising administering to the subject an inhibitor of PI3K (such as, for example, a VPS34 inhibitor including, but not limited to VPS34 IN1, VPS34 IN2 (commonly referred to as PIK-III), SB02024, compound 19, or SAR405 HY12481), an inhibitor of lipases and/or fatty acid synthase (such as, for example, Orlistat or C75), an inhibitor of fatty acyl-CoA-synthetases (such as, for example, Triacsin C), an inhibitor of diacylglycerol acyltransferase 1 (DGAT1) (such as, for example, A922500), a palmitoyl acyltransferases (PAT) inhibitor (such as, for example, 2-bromopalmitate), and/or an inhibitor of fatty acyl-CoA-carboxylase (such as, for example, 5-(Tetradecyloxy)-2-furoic acid (TOFA))). In one aspect, the inhibitor can be an antibody, siRNA, small molecule, peptide, or protein.

It is understood and herein contemplated that the therapeutic agents identified by the methods of screening disclosed herein can be used for the treatment of any virus and, in particular, coronaviruses. In one aspect, disclosed herein are methods of treating, reducing, inhibiting, decreasing, ameliorating and/or preventing a coronaviral infection of any preceding aspect, wherein the virus is hepatitis C virus; wherein the virus is a coronavirus selected from the group consisting of avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (BEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, delta variant, and/or P.1 variant), and middle east respiratory syndrome (MERS) coronavirus (CoV) (MERS-CoV); wherein the virus is a tombusvirus selected from the group consisting of artichoke mottled crinkle virus (AMCV), carnation Italian ringspot virus (CIRV), cucumber Bulgarian virus (CBLV), cucumber necrosis virus (CuNV), cumbidium ringspot virus (CymRSV), eggplant mottled crinkle virus (EMCV), grapevine Algerian latent virus (GALV), tomato bushy stunt virus (TBSV), Petunia asteroid mosaic virus (PetAMV), Pelargonium leaf curl virus (PLCV), Moroccan pepper virus (MPV), Limonium flower distortion virus (LFDV), Havel River virus (HRV), Lato river virus (LRV). Sitke waterborne virus (SWBV), and Neckar River virus (NRV); and/or wherein the virus is a rotavirus selected from the group consisting of rotavirus comprises rotavirus A, rotavirus B, rotavirus C, rotavirus D, rotavirus E, rotavirus F, rotavirus G, rotavirus H, rotavirus I, and rotavirus J.

In one aspect, disclosed herein are methods of inhibiting, reducing, decreasing, and/or preventing viral replication comprising contacting a virus with an inhibitor of fatty acid synthesis including but not limited to Orlistat, C75, protein palmitoylation inhibitors (such as, for example, a palmitoyl acyltransferases (PAT) inhibitor including, but not limited to 2-bromopalmitate), inhibitors of fatty acyl-CoA-synthetases (ACS)(such as, for example, Triacsin C), inhibitors of diacylglycerol acyltransferase 1 (DGAT1)(such as, for example, A922500), and/or inhibitors of fatty acyl-CoA-carboxylases (ACC)(such as, for example, TOFA).

Also disclosed herein are methods of inhibiting, reducing, decreasing, and/or preventing viral replication, wherein the virus is a coronavirus selected from the group consisting of avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (BEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, delta variant, and/or P.1 variant), and middle east respiratory syndrome (MERS) coronavirus (CoV) (MERS-CoV; hepatitis C virus; a tombusvirus selected from the group consisting of artichoke mottled crinkle virus (AMCV), carnation Italian ringspot virus (CIRV), cucumber Bulgarian virus (CBLV), cucumber necrosis virus (CuNV), cumbidium ringspot virus (CymRSV), eggplant mottled crinkle virus (EMCV), grapevine Algerian latent virus (GALV), tomato bushy stunt virus (TBSV), Petunia asteroid mosaic virus (PetAMV), Pelargonium leaf curl virus (PLCV), Moroccan pepper virus (MPV), Limonium flower distortion virus (LFDV), Havel River virus (HRV), Lato river virus (LRV). Sitke waterborne virus (SWBV), and Neckar River virus (NRV; or a rotasvirus selected from the group consisting of rotavirus A, rotavirus B, rotavirus C, rotavirus D, rotavirus E, rotavirus F, rotavirus G, rotavirus H, rotavirus I, and rotavirus J.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in °C or is at ambient temperature, and pressure is at or near atmospheric.

### 1. Example 1: Inhibitors of VPS34 and Lipid Metabolism Suppress SARS-CoV-2 Replication

SARS-CoV-2 is a beta-coronavirus, enveloped positive-sense, RNA virus, causing a pandemic for which there is an urgent need to understand virus replication requirements and identify therapeutic strategies. Repurposing drugs developed for other purposes can provide a shortcut to therapeutic development. Coronavirus (CoV) replication involves multiple critical interactions with host cell membranes, including viral entry, establishment of replication centers in double membrane vesicles, and virus release.

To assess whether SARS-CoV-2 is susceptible to modulators of membrane metabolism/biology we assessed the sensitivity of the virus in Vero E6 and Calu-3 cells to inhibitors of VPS34, Triacsin C which inhibits de novo synthesis of triacylglycerol, diacylglycerol and cholesterol esters, and Orlistat an inhibitor of lipases and fatty acid synthase (FASN). Two inhibitors of VPS34 potently inhibit SARS-CoV-2 replication, whereas an FDA-approved inhibitor of a different class of PI3K had minimal effect on replication. Targeting FASN and de novo synthesis of triacylglycerol, diacylglycerol and cholesterol esters each impairs SARS-CoV-2 replication. These studies therefore identify pathways as therapeutic targets and avenues for repurposing of existing drug candidates for SARS-CoV-2 infection.

### A) Materials and Methods

### Virus and Cell Lines

Vero E6 (ATCC# CRL-1586), Calu-3 (ATCC# HTB-55), and Caco-2 (ATCC# HTB-37) were maintained in DMEM (Corning) supplemented with 10% heat inactivated fetal bovine serum (FBS; GIBCO). Cells were kept in a 37° C., 5% CO2 incubator without antibiotics or antimycotics. SARS-CoV-2, strain USA_WA1/2020, was obtained from the World Reference Collection for Emerging Viruses and Arboviruses at the University of Texas Medical Branch-Galveston.

### Virus Propagation and Plaque Assays

A lyophilized ampule of SARS-CoV-2 was initially resuspended in DMEM supplemented with 2% FBS. VeroE6 cells were inoculated in duplicate with a dilution of 1:100 with an adsorption period of 1 hour at 37° C. and shaking every 15 minutes. Cells were observed for cytopathic effect (CPE) every 24 hours. Stock SARS-CoV-2 virus was harvested at 72 hours post infection (h.p.i) and supernatants were collected, clarified, aliquoted, and stored at -80° C.

92. Vero E6 cells were seeded onto a 24-well plate 24 hours before infection. 100ul of SARS-CoV-2 serial dilutions were added, adsorbed for 1 hour at 37C with shaking at 15-minute intervals. After the absorption period, 1 mL of 0.6% microcrystalline cellulose (MCC; Sigma 435244) in serum-free DMEM was added. To stain plaque assays MCC was aspirated out, 10% neutral buffered formalin (NBF) added for one hour at room temp and then removed. Monolayers were then washed with water and stained with 0.4% crystal violet. Plaques were quantified and recorded as PFU/mL.

### Confocal Microscopy

For confocal microscopy analysis, all cell lines were pre-seeded 24 hours before infection onto glass coverslips and infected with SARS-CoV-2 at an MOI of 1. At 24 h.p.i. supernatant was removed and samples fixed with 10% NBF for 1 hour at room temperature followed by PBS wash and permeabilized with sterile filtered 0.1% Saponin in PBS. Cells were blocked with 0.1% Saponin in Fluorescent Blocker (ThermoFisher) for 1 hour at RT. Primary antibodies were added and incubated overnight at 4C. AlexaFluor488, 594, and 647 were used as secondaries and nuclei stained with DAPI. Samples were imaged on Zeiss LSM800 Confocal with Super Resolution AiryScan. Images were rendered in ZenBlue or Imaris Viewer 9.0.

### Maestro Z Impedance Instrument

Prior to cell plating, CytoView-Z 96-well electrode plates (Axion Biosystems, Atlanta, GA) were coated with 5 µg/mL human fibronectin (Corning) for 1 hr at 37C. After coating, fibronectin was removed and 100 µL of DMEM/10%FBS was added to each well. The plate was then docked into the Maestro Z instrument for electrode baselining. Vero E6 cells were then plated to confluency (~75,000 cells/well) in the coated CytoView-Z plates and left at room temperature for 1 hour to ensure even coverage of the well. Plates containing Vero E6 cells were then docked into the Maestro Z and transepithelial electrical resistance (TEER) measurements were allowed to stabilized for 24 hours at 37C/5% C02. For compound treatments, media was removed from wells of the CytoView-Z plates and 195 µL of pre-warmed DMEM/2%FBS was added with the indicated concentration of compound. Infections with SARS-CoV-2 at an MOI of 0.01 were carried out by directly adding 5 µL of virus to each well. Plates were then docked within the Maestro-Z and TEER measurements were continuously recorded for 48-72 hours post-infection. All plates contained media only, full lysis, uninfected, and SARS-CoV-2 infected controls. For calculation of percent inhibition, raw TEER values were normalized to the uninfected control within the Axis Z software, and percent inhibition was calculated with the following formula: Percent Inhibition = 100*(1-(1- average of treated cells)/(1-average of infected control)). Median time to death calculations were performed by fitting the Boltzman sigmoid equation to raw kinetic TEER data. V50 values were used to determine median time to death.

### Cell Viability Assay

VeroE6 or Calu-3 cells were seeded in 96-well black walled microplates and incubated overnight. Cells were then treated with compounds and CellTox Green Dye (Promega) to monitor compound cytotoxicity. Fluorescence (Excitation: 485 nm, Emission: 520 nm) was measured every 24 hours post treatment for 3 days. Percent viability was determined using the minimum fluorescence obtained from media only cells and the maximum value obtained by cells lysed with 1% Triton-X.

### Labeling of Nascent Viral RNA

96. VeroE6 cells were seeded onto glass coverslips and incubated overnight at 37C. Cells were then infected with SARS-CoV-2 at an MOI of 3. At 24 h.p.i. cells were treated with 1 nM of Actinomycin D (Sigma) for 1 hour. Nascent RNA was labeled using Click-iT™ RNA Alexa Fluor™ 594 Imaging Kit (ThermoFisher). Cells were then processed for confocal analysis.

### Compounds

VPS34 IN-1 (#17392), PIK-III (#17002), Triacsin C (#10007448), Orlistat (#10005426), TOFA (#10005263), C75 (#10005270), Etomoxir (#11969), Trimetazidine (#18165), and A-922500 (#10012708) were purchased from Cayman Chemical (Ann Arbor, Michigan, USA). Remdesivir was purchased from Target Molecule Corp. (T7766, Boston, Massachusetts, USA). 2-bromopalmitate was purchased from Sigma-Aldrich (#21604, St. Louis, MO, USA). All compounds were resuspended in dimethylsulfoxide (DMSO).

### Quantification of vRNA and mRNA

Calu-3 cells were seeded in 24-well plates and allowed to grow to confluency. Infection with SARS-CoV-2 was carried out at an MOI of 0.01 and 1.0. Media containing the indicated compound was added 2 hours post infection. Supernatants were collected at 24 hours post infection for titering, and RNA was extracted from cell monolayers using TRIzol reagent (ThermoFisher). Post extraction, RNA was DNase treated using ezDNase (ThermoFisher, Waltham, MA, USA) and subjected to first strand synthesis using SuperScript IV (ThermoFisher, Waltham, MA, USA) using the included random hexamer primers. qPCR was performed using PerfeCTa CYBR Green FastMix (VWR, Radnor, PA, USA) and primers for SARS-CoV-2 N (genomic and subgenomic), SARS-CoV-2 NSP14, and RPS11 as an internal control. Each assay was performed in triplicate with three technical replicates, and each assay contained no-template controls. Data were analyzed by the ΔΔCt method with RPS11 serving as the housekeeping gene and uninfected DMSO treated Calu3 cells as a mock control.

### sgRNA Selection

sgRNAs were designed according to Synthego’s multi-guide gene knockout. Briefly, two or three sgRNAs are bioinformatically designed to work in a cooperative manner to generate small, knockout-causing, fragment deletions in early exons. These fragment deletions are larger than standard indels generated from single guides. The genomic repair patterns from a multi-guide approach are highly predictable based on the guide-spacing and design constraints to limit off-targets, resulting in a higher probability protein knockout phenotype.

### sgRNA Synthesis

RNA oligonucleotides were chemically synthesized on the Synthego solid-phase synthesis platform, using CPG solid support containing a universal linker. 5-Benzylthio-1H-tetrazole (BTT, 0.25 M solution in acetonitrile) was used for coupling, (3-((Dimethylamino-methylidene)amino)-3H-1,2,4-dithiazole-3-thione (DDTT, 0.1 M solution in pyridine) was used for thiolation, dichloroacetic acid (DCA, 3% solution in toluene) for used for detritylation. Modified sgRNA were chemically synthesized to contain 2′-O-methyl analogs and 3′ phosphorothioate nucleotide interlinkages in the terminal three nucleotides at both 5′ and 3′ ends of the RNA molecule. After synthesis, oligonucleotides were subject to series of deprotection steps, followed by purification by solid phase extraction (SPE). Purified oligonucleotides were analyzed by ESI-MS.

### RNP Formation and Transfection

To deliver CRISPR-Cas9 ribonucleoprotein (RNP) complexes, 10 pmol Streptococcus pyogenes NLS-Sp.Cas9-NLS (SpCas9) nuclease (Aldevron Cat. #9212) was combined with 30 pmol total synthetic sgRNA (Synthego, 10 pmol each sgRNA) to form RNPs in 20 uL total volume with SF Buffer (Lonza Cat #V5SC-2002) and allowed to complex at room temperature for 10 minutes. Cells were dissociated into single cells using TrypLE Express (Gibco), as described above, resuspended in culture media and then counted. 100,000 cells per nucleofection reaction were pelleted by centrifugation at 100 xg for 3 minutes. Following centrifugation, cells were resuspended in transfection buffer and diluted to 2^{∗}10⁴ cells/µL. 5 µL of cell solution was added to preformed RNP solution and gently mixed. Nucleofections were performed on a Lonza 96-well nucleofector shuttle system using program CM-150. Immediately following nucleofection each reaction was transferred to a tissue-culture treated 96-well plate containing 100 µL normal culture media and seeded at a density of 50,000 cells per well. Transfected cells were incubated following standard protocols.

### Genomic Analysis

Two days post-nucleofection, DNA was extracted from using DNA QuickExtract (Lucigen Cat. #QE09050). Briefly, cells were lysed by removal of the spent media followed by addition of 50 µL of QuickExtract solution to each well. Once the QuickExtract DNA Extraction Solution was added, the cells were scraped off the plate into the buffer. Following transfer to compatible plates, DNA extract was then incubated at 68° C. for 15 minutes followed by 95° C. for 10 minutes in a thermocycler before being stored for downstream analysis.

103. Amplicons for indel analysis were generated by PCR amplification AmpliTaq Gold 360 polymerase (Thermo Fisher Scientific Cat. #4398881) according to the manufacturer’s protocol. Primers were designed to create amplicons between 400 - 800 bp, with both primers at least 100 bp distance from any of the sgRNA target sites. PCR products were cleaned-up and analyzed by Sanger sequencing (Genewiz). Sanger data files and sgRNA target sequences were input into Inference of CRISPR Edits (ICE) analysis (ice.synthego.com) to determine editing efficiency and to quantify generated indels. Percentage of alleles edited is expressed an ice-d score. This score is a measure of how discordant the Sanger trace is before versus after the edit. It is a simple and robust estimate of editing efficiency in a pool, especially suited to highly disruptive editing techniques like multi-guide.

### Supplementation

Supplementation with fatty acids and lipids was achieved by pre-complexing the supplements to fatty acid free bovine serum albumin (FAF-BSA). Briefly, FAF-BSA was dissolved in tissue culture grade water to achieve a final concentration of 10%. Stock solutions of palmitic acid (150 mM in ethanol, Sigma-Aldrich St. Louis, MO, USA) and oleic acid (3 M, Sigma-Aldrich St. Louis, MO, USA) were diluted to 2.5 mM in 10% FAF-BSA and incubated at 37° C. with frequent mixing for 30 minutes. The pre-complexed solutions were then added to DMEM at a final concentration of 2% FAF-BSA and 0.25 mM supplement. For the infection, NT-WT and FASN KO Caco2 cells were pre-seeded in 96 well plates and allowed to grow to confluency. Infection with SARS-CoV-2 was performed at an MOI of 0.01 in the serum-free media. One hour post adsorption, virus inoculum was removed and medium containing the designated serum/lipid treatment was added. Cells were incubated at 37C with 5% CO₂ until the designated end point. Supernatants were collected and quantified by plaque assay.

### GFP-2xFYVE Assay

Huh7 cells were seeded in 96-well black walled plates and transfected with a pEGFP-2xFYVE plasmid obtained from Addgene (#140047) using Lipofectamine 2000 (Invitrogen St. Louis, MO, USA). 24 hours post transfection (hpt) media was removed and replaced with media containing either DMSO, VPS34-IN1 (2 µM), PIK-III (5 µM), SAR405 (5 µM), Compound 19 (5 µM), or Orlistat (200 µM). 24 hours post treatment cells were imaged using a BioTek Cytation 5 to determine the localization pattern of eGFP-2xFYVE.

### B) Results

### Development of 96-Well Format Impedance-based Assay to Measure SARS-CoV-2 Cytopathic Effects

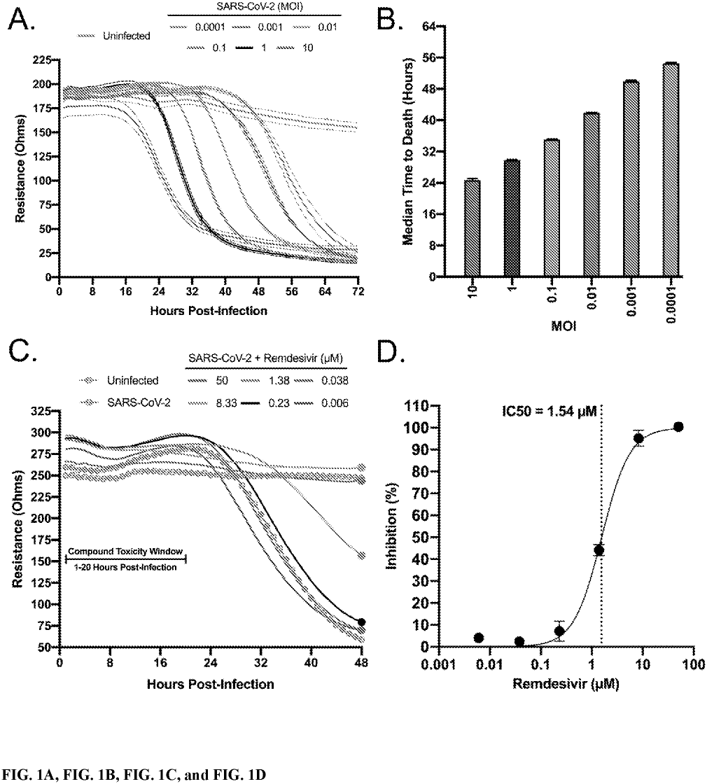

SARS-CoV-2 induces significant cytopathic effects in infected Vero E6 cells. Based on this property, we standardized a 96-well format assay that provides continuous real-time, label free monitoring of the integrity of cell monolayers as a direct correlation of virus growth and infection. This assay was standardized using the Maestro Z platform (Axion BioSystems, Atlanta, GA), an instrument that uses plates containing electrodes in each well (CytoView-Z plates) to measure electrical impedance across the cell monolayer every minute throughout the course of the experiment. As SARS-CoV-2 induced cytopathic effects damage the cell monolayer, impedance measurements decrease over time providing a detailed assessment of infection kinetics. To determine the capacity of the system to differentiate levels of virus replication, confluent Vero E6 monolayers in CytoView-Z plates were infected with SARS-CoV-2 at multiple MOIs (0.0001 to 10) and resistance measurements were acquired for 72 hours post infection (FIG. 1A). The progression of infection at each MOI was clearly distinct. A decrease in resistance could be observed as early as 18-20 hours post-infection (hpi) at an MOI of 1 and 10, and as late as 56 hpi at an MOI of 0.0001. All sample signals reached their lowest point between 32 to 72 hpi in an MOI dependent manner. The raw kinetic data was used to determine the median time to cell death for each MOI which shows a direct correlation with a decrease in resistance (FIG. 1B). MOI of 0.01 was chosen for antiviral assays based on its desirable infection kinetics.

To establish the Maestro Z as a potential instrument for screening of anti-SARS-CoV-2 therapeutics, we first tested Remdesivir, a well-described inhibitor of SARS-CoV-2 that has been granted emergency use authorization (EUA) for the treatment of COVID-19. Vero E6 cells were seeded on a CytoView-Z plate, incubated overnight to allow cells to stabilize, pretreated with 6-fold dilutions of Remdesivir for 1 hour and infected with SARS-CoV-2. Resistance measurements were recorded for 48 hpi (FIG. 1C). We determined a 50% inhibitory concentration (IC50) for Remdesivir of 1.54 µM (FIG. 1D). Taken together, these data validate the impedance-based assay described as a tool for screening of potential SARS-CoV-2 therapeutics.

### Inhibitors of VPS34 Activity Impair SARS-CoV-2 Growth

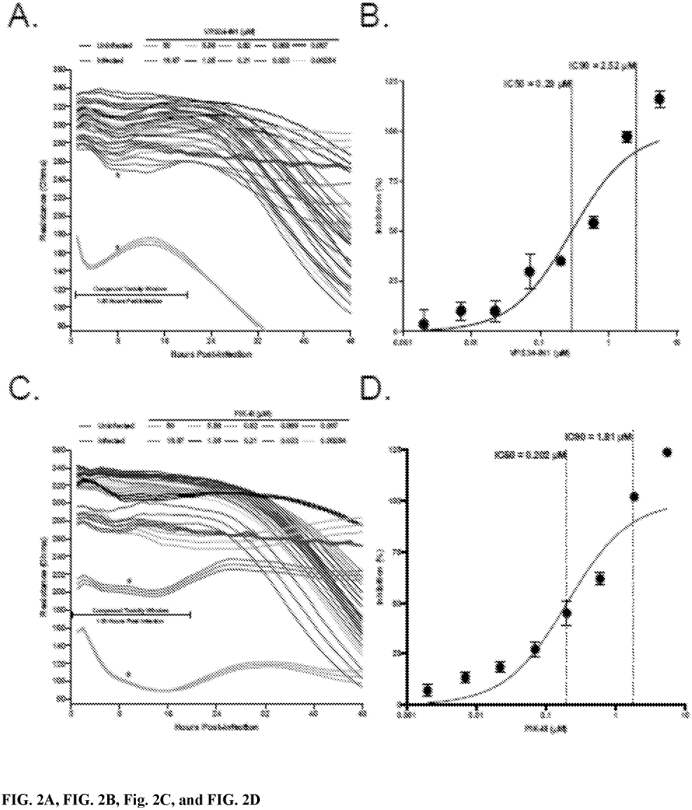

VPS34 is a multifunctional protein involved in autophagy and membrane trafficking. On account of the significant role of membrane rearrangements in coronavirus replication, we wanted to determine if VPS34 activity was essential for SARS-CoV-2 growth. We tested two well characterized VPS34 inhibitors, VPS34-IN1 and PIK-III. This was done over a 10-point dose response using the resistance assay described above. Briefly, pre-plated Vero E6 cells were treated with compound 1 hour prior to infection with SARS-CoV-2. Both VPS34-IN1 and PIK-III induced rapid cytotoxicity at 50 µM and 16.67 µM as indicated by a rapid decrease in resistance measurements between 1 and 20 hpi. (FIGS. 2A and 2C). At the remaining concentrations, no toxicity was observed. For several concentrations, the integrity of the monolayer was preserved relative to the mock-treated infected control indicating an antiviral effect of both VPS34-IN1 and PIK-III. Based on normalized resistance measurements at 48 hpi for non-toxic doses, we estimated IC50 values of <600 nM for VPS34-IN1 and PIK-III (FIGS. 2B and 2D, respectively). Additionally, IC90s of 2.52 µM (VPS34-IN1) and 1.81 µM (PIK-III) were also calculated. These data indicate that the VPS34 kinase plays a significant role in SARS-CoV-2 replication.

### Inhibition of Fatty Acid Metabolism Impairs SARS-CoV-2 Growth

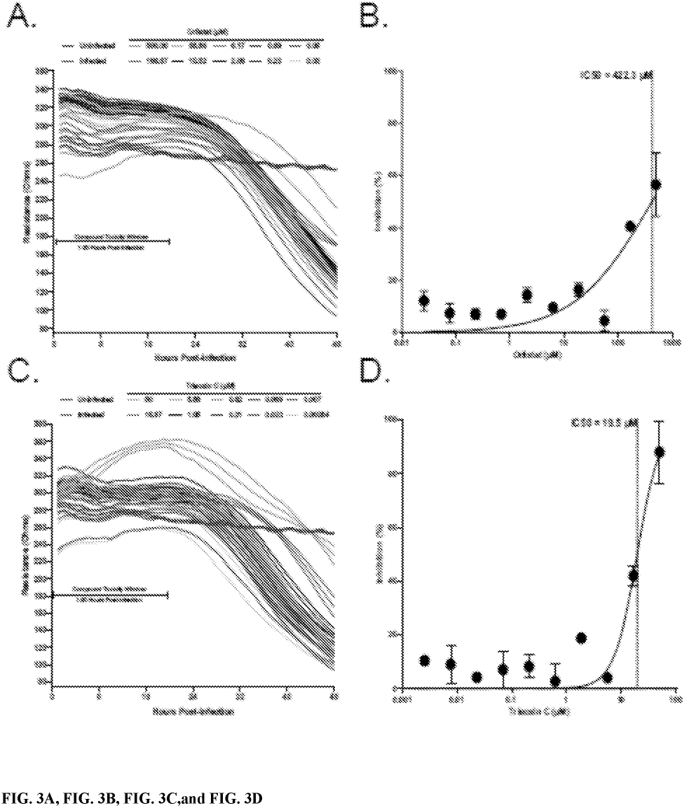

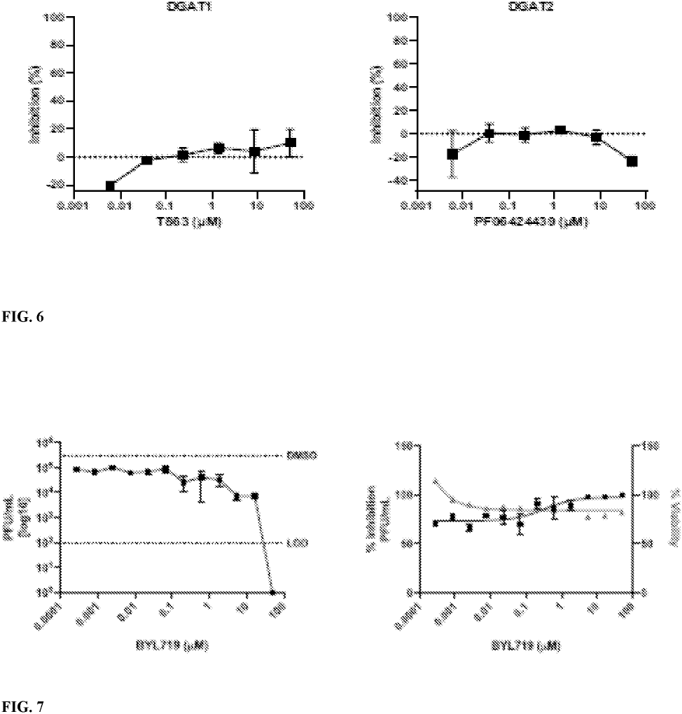

Fatty acid metabolism contributes to various host processes including production of lipid-based molecules such as triglycerides, phospholipids, and cholesterol, as well as protein modifications, such as palmitoylation and myristoylation. Modulation of fatty acid metabolism has been shown to impact replication and virion maturation for numerous flaviviruses, enteroviruses, and alphaviruses. Two well-described compounds that inhibit fatty acid metabolism are Orlistat, an FDA-approved drug that inhibits gastric lipases and fatty acid synthase (FASN), and Triacsin C, an inhibitor of long chain Acyl-CoA synthetase (ACS), both of which have been shown to have antiviral activity. To test the efficacy of these compounds against SARS-CoV-2 infection, Vero E6 cells were pre-seeded onto a CytoView-Z plate, allowed to stabilize, and then pre-treated with Orlistat or Triacsin C for 1 hour prior to infection. Based on the toxicity window of 1-20 hours post-treatment (hpt) determined with the VPS34 inhibitors, neither Orlistat nor Triacsin C induced early cytotoxic effects, even at the highest concentrations of 50 µM and 500 µM, respectively (FIGS. 3A and 3C). Both compounds exhibited inhibition of viral cytopathic effects at higher concentrations, although complete inhibition was not achieved even with 500 µM of Orlistat. Based on normalized resistance measurements at 48 hpi, we estimated IC50s of ~500 µM for Orlistat and ~20 µM for Triacsin C (FIGS. 3B and 3D). Viruses such as HCV and rotavirus that are sensitive to inhibition by Triacsin C are also impaired by inhibitors of DGATs. Therefore, we tested the effects of DGAT1 and DGAT2 inhibitors T863 and PF06424439. Neither compound displayed any inhibitory activity (FIG. 6). This data indicates that fatty acid metabolism and neutral lipid synthesis likely play an important role in SARS-CoV-2 infection.

### VPS34 and Fatty Acid Metabolism Inhibitors Exhibit Activity on Post-Entry Steps of the Viral Life Cycle

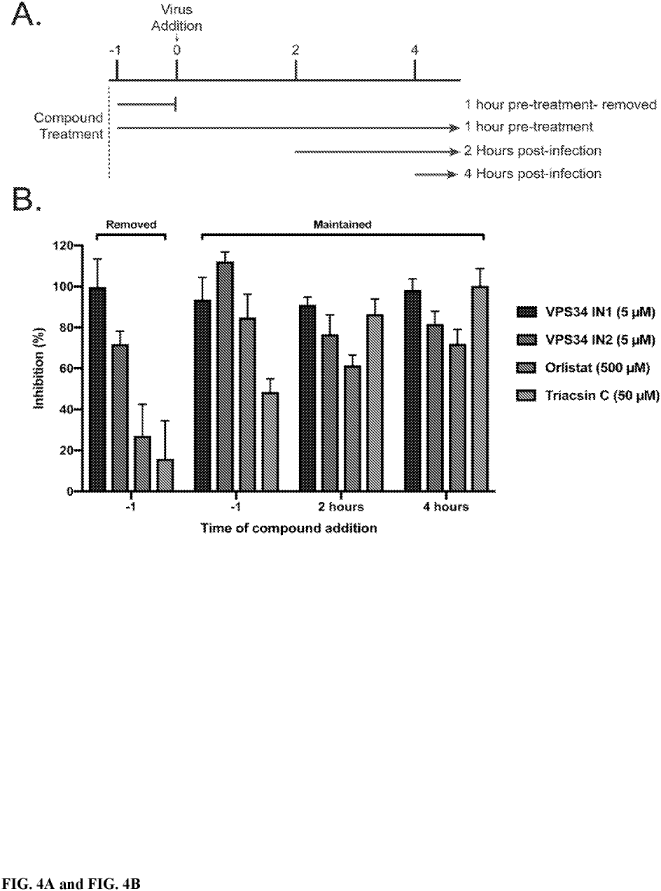

Next, we performed time of addition studies with VPS34-IN1, PIK-III, Orlistat, or Triacsin C. This allowed for the identification of the anti-viral activity of each compound impacted the viral life cycle at steps pre- or post- entry. As indicated in FIG. 4A, three conditions were tested: 1) single treatment 1 hour prior to viral infection, with compound removed prior to infection; 2) dosing at 2 hpi; and 3) dosing at 4 hpi. We observed that a single 5 µM treatment of VPS34-IN1 or PIK-III prior to infection inhibited SARS-CoV-2 replication, and inhibition could be observed even when added up to 4 hpi (FIG. 4B). In contrast, Orlistat or Triacsin C showed minimal efficacy when removed prior to infection but remained inhibitory when added up to 4hpi. Altogether, these data demonstrate that the VPS34 inhibitors likely act on viral entry and at later steps in the replication cycle; and inhibition by Orlistat and Triacsin C occurs post-entry.

### Attenuation of VPS34 Kinase Activity and Fatty Acid Metabolism Inhibit SARS-CoV-2 in a Human Airway Epithelial Cell Line

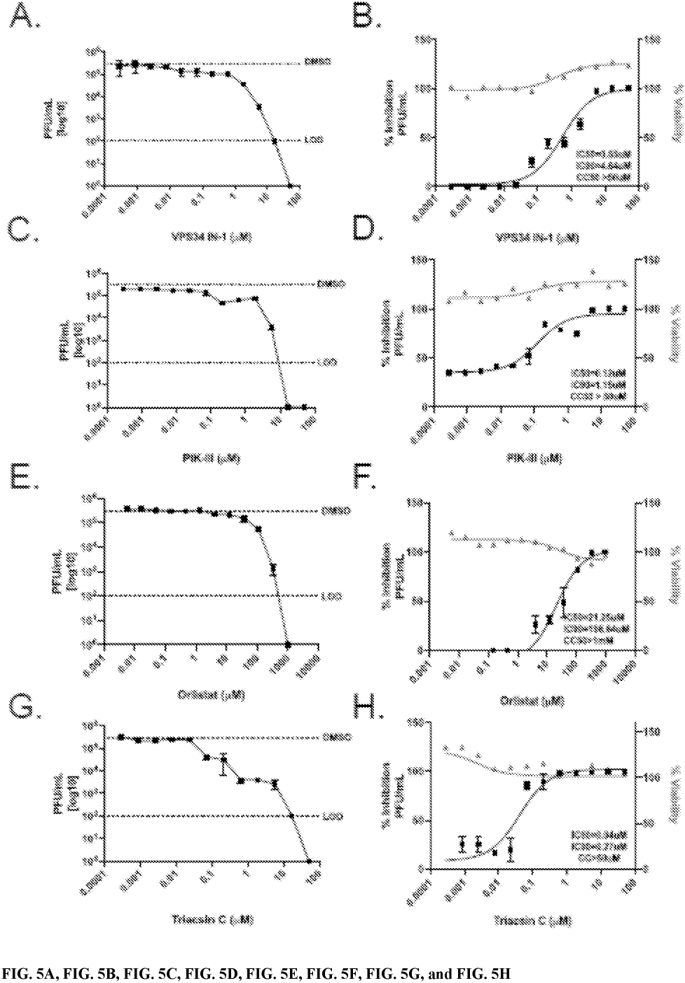

To determine whether or not the inhibition could be maintained in a cell line more functionally relevant to human infection by SARS-CoV-2, we directly measured the impact of compounds on production of infectious virus and cell viability in Calu-3 cells. These cells are derived from human airway epithelium and are highly susceptible to infection, establishing them as a standard for infection studies with SARS-CoV-1, MERS-CoV and SARS-CoV-2 (including, but not limited to the B1.351 variant, B.1.1.7 variant, delta variant, and/or P.1 variant). For these studies, Calu-3 cells were plated onto 96-well plates and allowed to reach confluency. Cells were either pre-treated with a range of concentrations of VPS34-IN1, PIK-III, Triacsin C, Orlistat, and DMSO, or mock treated with media alone for 1 hour, then infected with SARS-CoV-2 at an MOI of 0.01. Supernatants were collected at 48 hpi and viral titer determined by plaque assay. Cytotoxicity of the compounds was determined in parallel. Calu-3 cells were seeded onto 96-well black walled 96-well plates, allowed to reach 95% confluency and treated with VPS34-IN1, PIK-III, Triacsin C, Orlistat, DMSO, or mock treated with media alone. CellTox Green was added at the time of dosing and fluorescence measured at 48 h.p.i. in order to assess cytotoxicity. Consistent with results from infected Vero E6 cells, each of the compounds tested above inhibited production of infectious virus (FIGS. 5A, 5C, 5E, and 5G) and cytotoxicity was only observed in Calu-3 cells at the highest concentrations of some compounds. We observed IC50s of 0.55 µM (VPS34-IN1; FIG. 5B), 0.12 µM (PIK-III; FIG. 5D), 21.25 µM (Orlistat; FIG. 5F), and 0.04 µM (Triacsin C; FIG. 5H). Notably, the IC50s measured for VPS34-IN1 and PIK-III by plaque assay are in close agreement with estimated IC50s determined in Vero E6 cells using the resistance-based assay. The IC50s for Orlistat and Triacsin C were substantially lower than those estimated in VeroE6 cells.

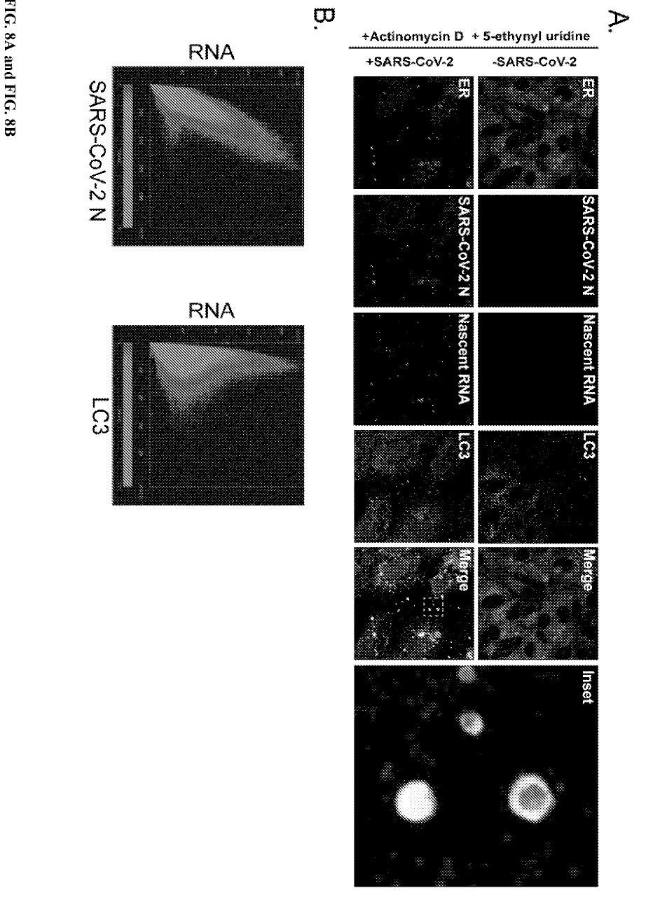

VPS34 is a class III PI3 kinase. We therefore extended our study to determine if BYL719, an FDA approved inhibitor of class I PI3 kinase used to treat breast cancer, would also inhibit SARS-CoV-2 replication in Calu-3 cells. Unlike the VPS34-specific inhibitors, little inhibition was detected up to 16.6 µM, at which we observed a 1-log decrease in viral titers (FIG. 7). This data indicates that not all PI3K classes play a significant role during SARS-CoV-2 replication.

To determine the specificity of inhibition to the class III PI3 kinase VPS34, we extended the Calu-3 compound study to include two additional VPS34 inhibitors: SAR405 and compound 19. SAR405 and compound 19 each inhibited with IC50s of 0.376 µM and 5.843 µM, respectively. This data further supports the importance of the class III PI3 kinase during SARS-CoV-2 replication. To confirm that the compounds were able to inhibit VPS34 activity, we assessed in Huh7 cells the effect of the inhibitors on GFP-2xFYVE localization to endosomes, indicating a loss of phosphoinositol-3 phosphorylation by VPS34, Huh7 cells were chosen for their favorable characteristics, such as transfectability and large cytoplasm that facilitates imaging. While DMSO or Orlistat did not affect GFP-2xFYVE localization, the four VPS34 inhibitors disrupted GFP-2xFYVE puncta, consistent with loss of PI-3 phosphorylation.

### Inhibition of VPS34 Kinase Activity and Fatty Acid Alters SARS-CoV-2 Replication Centers.

SARS-CoV-1 and MERS-CoV replicate in double membrane compartments to which the autophagy membrane marker LC3 localizes. We investigated if, similar to SARS-CoV-1 and MERS, SARS-CoV-2 nascent viral RNA and N co-localized with LC3. VeroE6 cells were infected with SARS-CoV-2 at a MOI of 3 and at 24 h.p.i., were treated with 1 µM of actinomycin D to arrest host-cell transcription. Cells where then chased for 4 hours with 5-ethynyl uridine (EU). Viral nascent RNA labeled during the EU chase was then detected with click chemistry, indirect immunofluorescence performed using primary antibodies against N and LC3, and the endoplasmic reticulum (ER) was detected with DPX BlueWhite ER stain. We observed distinct formation of ring-like structures positive for ER, N, LC3, and nascent viral RNA (FIG. 8A). Co-localization analysis demonstrated that nascent viral RNA co-localized with N or LC3 (FIG. 8B). This data demonstrates the presence of SARS-CoV-2 replication centers that form in association with LC3.

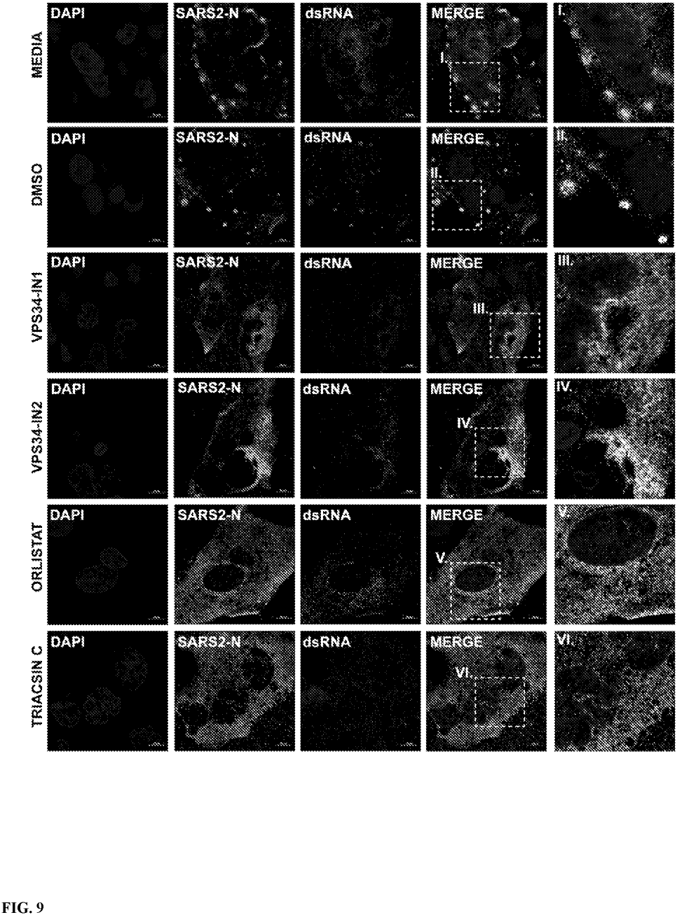

Because each compound exhibited inhibitory effects when added after viral entry, we sought to investigate through confocal microscopy, whether the compounds altered the establishment of viral replication centers, which form in association with double membrane structures. Calu-3 cells were seeded onto fibronectin coated glass cover slips and allowed to reach 95% confluency. Cells were pre-treated with approximately the IC90 of VPS34-IN1 (5uM), VPS34-IN2 (5 uM), Orlistat (500 uM), Triacsin C (50 uM), TOFA, 2-bromopalmitate, A922500, or Remdesivir and infected with SARS-CoV-2 at a MOI of 3. At 24 h.p.i. cells were fixed, permeabilized, and indirect immunofluorescence performed using primary antibodies against SARS-CoV-2 nucleoprotein (N) and dsRNA. We observed that when compared to the media only or DMSO control, N became completely cytoplasmic and did not form any large inclusion like formations in the presence of the compounds (FIG. 9). Additionally, even though dsRNA can be detected both distributed throughout the cytoplasm and associated with N in large inclusion like formations in the media only and DMSO control, in the cells treated with inhibitors, dsRNA was solely cytoplasmic. In particular, treatments with VPS34-IN1, Orlistat, TOFA, or A922500 resulted in a loss of these distinct puncta and dispersion of dsRNA and SARS-CoV-2 N. When treated with Triacsin C and 2-bromopalmitate, cells exhibited fewer but larger foci positive for both markers. Treatment with a non-sterilizing concentration of Remdesivir (1 µM) served as a control and yielded a distribution of N and dsRNA similar to the DMSO control. Thus, replication center formation was disrupted.

### Protein Palmitoylation and Triacylglycerol Production are Implicated in SARS-CoV-2 Infection

Several recent reports have identified that pathways related to lipid metabolism and cholesterol homeostasis are required for SARS-CoV-2 replication; however, the precise mechanism by which these pathways contribute to the SARS-CoV-2 replication cycle remains unclear. Given that the data indicates a role for fatty acid metabolism, we set out to further clarify the enzymatic steps required for SARS-CoV-2 replication.

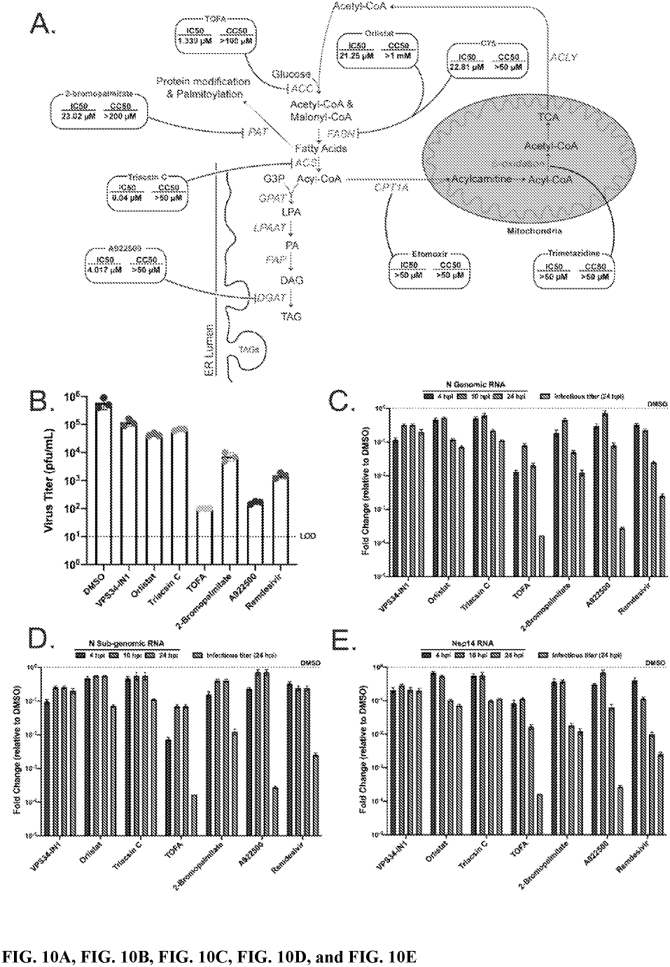

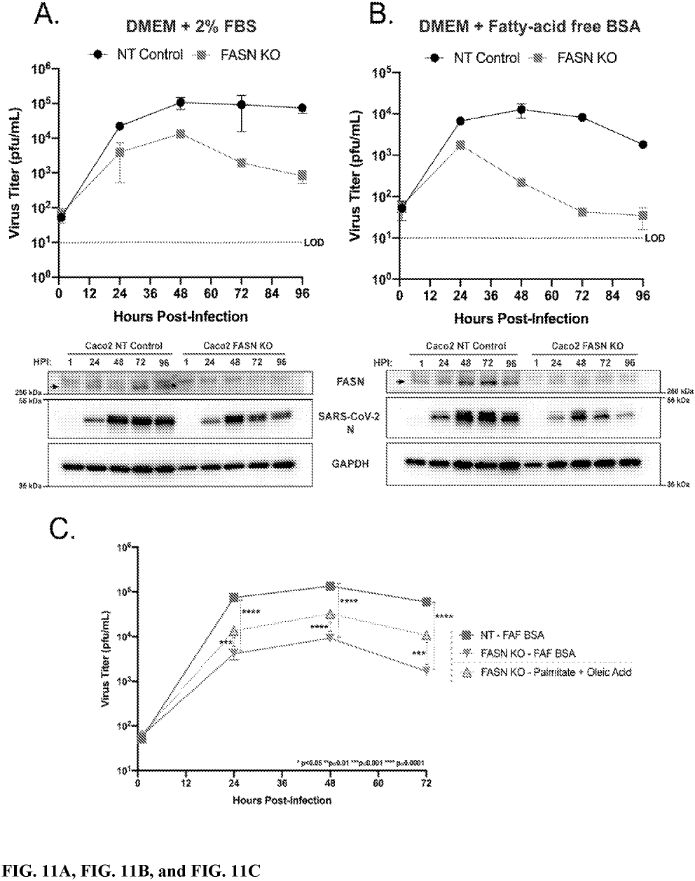

To further evaluate the importance of de novo fatty acid synthesis, TOFA, a competitive inhibitor of acetyl-CoA carboxylase (ACC), the enzyme directly upstream of FASN, and tetrahydro-4-methylene-2R-octyl-5-oxo-3S-furancarboxylic acid (C75), an additional inhibitor of FASN, were used. Both compounds exhibited activity against SARS-CoV-2 infection with IC50s of 1.339 µM and 22.81 µM respectively (FIG. 10).

Inhibition of fatty acid synthesis at early enzymatic steps like FASN can lead to dysregulation of three main downstream pathways: fatty acid based protein modification, fatty acid β-oxidation in the mitochondria, and neutral lipid synthesis. To further determine which branches and enzymatic steps of fatty acid metabolism contribute to anti-SARS-CoV-2 activity, a series of additional compounds were tested for inhibitory activity and cytotoxicity (FIG. 10).

To assess the contribution of decreased protein palmitoylation, 2-bromoplamitate, an inhibitor of palmitoyl acyltransferases (PAT), was used. 2-bromopalmitate inhibited infectious virus production at concentrations greater than 10 µM with an IC50 of 23.02 µM (FIG. 10A). To determine if fatty acid β-oxidation in the mitochondria contributes to inhibition of SARS-CoV-2, two compounds were tested: Etomoxir, a compound that targets carnitine palmitoyltransferase 1A (CPT1A), blocking translocation of fatty acids into the mitochondria, and Trimetazidine, an inhibitor of long-chain 3-ketoacyl-CoA thiolase. Neither compound showed any inhibition, indicating that fatty acid β-oxidation is not required for SARS-CoV-2 replication (FIG. 10A). Lastly, the importance of the terminal steps of the fatty acid metabolism pathways was assessed by inhibiting neutral lipid production and lipid droplet formation using A922500, a potent inhibitor of diacylglycerol acyltransferase 1 (DGAT1). Treatment with A922500 potently inhibited SARS-CoV-2 with an IC50 value of 4.017 µM (FIG. 10A). All together, these data indicate that protein palmitoylation and neutral lipid synthesis are needed for efficient SARS-CoV-2 replication.

### Inhibition of VPS34 and Fatty Acid Metabolism Impacts Genomic and Subgenomic RNA Levels

The effects on dsRNA and N distribution indicated possible effects on viral RNA synthesis. To determine if the production of viral RNA was affected, Calu-3 cells were infected at an MOI of either 0.01 or 1. Two hours post-infection, cells were treated with DMSO or concentrations of each inhibitor determined to result in significant inhibition at an MOI of 0.01 without toxicity. At 4, 10, and 24 hpi, levels of genomic and subgenomic N and NSP14 RNA were quantified by RT-PCR. Supernatants corresponding to 24 hpi were used to quantify viral titers to ensure inhibition was achieved. Remdesivir was used as a positive control compound that inhibits SARS-CoV-2 RNA synthesis.

Compound treatments resulted in a 1- to 4-log₁₀ reduction of viral titers at both MOIs at 24 hpi (FIG. 10B). Consistent with the decrease in viral titers, all compounds reduced the amounts of genomic RNA at the 24 h time point. Remdesivir inhibited RNA synthesis at earlier time points as well. At 4 and 10 hpi at MOI of 1, the inhibition was more apparent with the N genomic RNA and NSP14 primers as compared with the N sub-genomic RNA primers. The degree of inhibition was magnified at an MOI of 0.01 (FIGS. 10C-E). Similarly, treatment with VPS34-IN1, Orlistat, Triacsin C, and 2-bromopalmitate reduced RNA levels at both MOI 0.01 and 1. In general, there were minimal differences in fold-inhibition at 4 versus 10 hpi. (FIGS. 10C-E). The inhibition of RNA synthesis resulting from treatment with TOFA and A922500 followed a similar pattern to the other compounds at each MOI and time point. However, the reduction in RNA synthesis at 24 hpi was significantly less substantial than the reduction in viral titers. (FIGS. 10C-E). This discordance indicates that these compounds have additional effects beyond viral RNA synthesis that explain their inhibition of virus growth.

### Genetic Knockout Confirms a Critical Role of De Novo Fatty Acid Synthesis for SARS-CoV-2 Replication

As a genetic correlate to the studies described above, we compared the growth of SARS-CoV-2 in non-targeting control (NT) and FASN knockout Caco2 (FASN KO) cells in the presence of 2% FBS or 1% fatty acid free (FAF)-BSA. The latter condition addressed the possible contribution of exogenous fatty acids supplied by the FBS. Consistent with the compound data, SARS-CoV-2 replicated to substantially lower titers in the FASN KO cells as compared to WT cells when the cells were maintained in medium with 2% fetal bovine serum (FBS) (FIG. 11A). The inhibition was enhanced when cells were maintained in 1% FAF-BSA media, reaching an almost 2-log₁₀ reduction in titer as early as 48 hpi (FIG. 11B). For each experiment, FASN knockout was confirmed by western blot. In addition, SARS-CoV-2 N protein levels were decreased in FASN KO cells as compared to NT cells. At later timepoints, N protein levels in FASN KO cells decline consistent with the changes in virus titer. The diminished replication and protein production in cells lacking FASN confirms the earlier findings and indicates that fatty acid metabolism is necessary for efficient, sustained replication during SARS-CoV-2 infection.

Based on the observations that the reduction in protein palmitoylation and neutral lipid synthesis are likely responsible for the inhibition seen when blocking fatty acid metabolism, we asked whether viral replication in FASN knockout cells would be restored by supplementing with substrates that feed into these two sub-pathways. Supplementation was achieved by infecting FASN KO Caco2 cells in minimal media and adding back media containing 2% FAF-BSA pre-complexed to a combination of 250 µM palmitic acid and 250 µM oleic acid. Cell supernatants were collected at 1, 24, 48, and 72 hpi and viral titers were determined by plaque assay. Consistent with the data, growth in FASN KO cells was significantly reduced as compared to NT control cells. Supplementation with palmitate and oleic acid partially rescued virus growth further confirming the importance of fatty acid metabolism in SARS-CoV-2 replication (FIG. 11C).

### REFERENCES

Abu-Farha, M., Thanaraj, T.A., Qaddoumi, M.G., Hashem, A., Abubaker, J., and Al-Mulla, F. (2020). The Role of Lipid Metabolism in COVID-19 Virus Infection and as a Drug Target. Int J Mol Sci. 21(10).

Ackerman, D., Tumanov, S., Qiu, B., Michalopoulou, E., Spata, M., Azzam, A., Xie, H., Simon, M.C., and Kamphorst, J.J. (2018). Triglycerides Promote Lipid Homeostasis during Hypoxic Stress by Balancing Fatty Acid Saturation. Cell Rep. 24(10), 2596-2605 e2595.

Alsabeeh, N., Chausse, B., Kakimoto, P.A., Kowaltowski, A.J., and Shirihai, O. (2018). Cell culture models of fatty acid overload: Problems and solutions. Biochim Biophys Acta Mol Cell Biol Lipids. 1863(2), 143-151.

Ammer, E., et al., The anti-obesity drug orlistat reveals anti-viral activity. Med Microbiol Immunol, 2015. 204(6): p. 635-45.

Angelini, M.M., Akhlaghpour, M., Neuman, B.W., and Buchmeier, M.J. (2013). Severe acute respiratory syndrome coronavirus nonstructural proteins 3, 4, and 6 induce double-membrane vesicles. mBio. 4(4).

Backer, J.M. (2016). The intricate regulation and complex functions of the Class III phosphoinositide 3-kinase Vps34. Biochem J. 473(15), 2251-2271.

Baenke, F., Peck, B., Miess, H., and Schulze, A. (2013). Hooked on fat: the role of lipid synthesis in cancer metabolism and tumour development. Dis Model Mech. 6(6), 1353-1363

Bago et al. Characterization of VPS34-IN1, a Selective Inhibitor of Vps34, Reveals That the Phosphatidylinositol 3-phosphate-binding SGK3 Protein Kinase Is a Downstream Target of Class III Phosphoinositide 3-kinase. Biochem J. 2014 Nov 1;463(3):413-27.

Bakhache, W., et al., Fatty acid synthase and stearoyl-CoA desaturase-1 are conserved druggable coƒactors oƒ Old World Alphavirus genome replication. Antiviral Res, 2019. 172: p. 104642.

Bernasconi, R., J. Noack, and M. Molinari, Unconventional roles oƒ nonlipidated LC3 in ERAD tuning and coronavirus inƒection. Autophagy, 2012. 8(10): p. 1534-6.

Boscarino, J.A., Logan, H.L., Lacny, J.J., and Gallagher, T.M. (2008). Envelope protein palmitoylations are crucial for murine coronavirus assembly. J Virol. 82(6), 2989-2999.

Bouhaddou, M., Memon, D., Meyer, B., White, K.M., Rezelj, V.V., Correa Marrero, M., Polacco, B.J., Melnyk, J.E., Ulferts, S., Kaake, R.M., et al. (2020). The Global Phosphorylation Landscape of SARS-CoV-2 Infection. Cell. 182(3), 685-712 e619.

Carvalho, M.A., Zecchin, K.G., Seguin, F., Bastos, D.C., Agostini, M., Rangel, A.L., Veiga, S.S., Raposo, H.F., Oliveira, H.C., Loda, M., et al. (2008). Fatty acid synthase inhibition with Orlistat promotes apoptosis and reduces cell growth and lymph node metastasis in a mouse melanoma model. Int J Cancer. 123(11), 2557-2565.

Chen, B., Sun, Y., Niu, J., Jarugumilli, G.K., and Wu, X. (2018). Protein Lipidation in Cell Signaling and Diseases: Function, Regulation, and Therapeutic Opportunities. Cell Chem Biol. 25(7), 817-831.

Cheung, W., et al., Rotaviruses associate with cellular lipid droplet components to replicate in viroplasms, and compounds disrupting or blocking lipid droplets inhibit viroplasm ƒormation and viral replication. J Virol, 2010. 84(13): p. 6782-98.

Cloherty, A.P.M., Olmstead, A.D., Ribeiro, C.M.S., and Jean, F. (2020). Hijacking of Lipid Droplets by Hepatitis C, Dengue and Zika Viruses-From Viral Protein Moonlighting to Extracellular Release. Int J Mol Sci. 21(21).

Cottam, E.M., et al., Coronavirus nsp6 proteins generate autophagosomes from the endoplasmic reticulum via an omegasome intermediate. Autophagy, 2011. 7(11): p. 1335-47.

Daniloski, Z., Jordan, T.X., Wessels, H.H., Hoagland, D.A., Kasela, S., Legut, M., Maniatis, S., Mimitou, E.P., Lu, L., Geller, E., et al. (2021). Identification of Required Host Factors for SARS-CoV-2 Infection in Human Cells. Cell. 184(1), 92-105 e116.

Davda, D., El Azzouny, M.A., Tom, C.T., Hernandez, J.L., Majmudar, J.D., Kennedy, R.T., and Martin, B.R. (2013). Profiling targets of the irreversible palmitoylation inhibitor 2-bromopalmitate. ACS Chem Biol. 8(9), 1912-1917.

Dias, S.S.G., Soares, V.C., Ferreira, A.C., Sacramento, C.Q., Fintelman-Rodrigues, N., Temerozo, J.R., Teixeira, L., Nunes da Silva, M.A., Barreto, E., Mattos, M., et al. (2020). Lipid droplets fuel SARS-CoV-2 replication and production of inflammatory mediators. PLoS Pathog. 16(12), e1009127.

Dowdle, W., Nyfeler, B., Nagel, J. et al. Selective VPS34 inhibitor blocks autophagy and uncovers a role for NCOA4 in ferritin degradation and iron homeostasis in vivo. Nat Cell Biol 16, 1069-1079 (2014).

Feng, Z., et al., Recruitment oƒ Vps34 PI3K and enrichment oƒ PI3P phosphoinositide in the viral replication compartment is crucial ƒor replication oƒ a positive-strand RNA virus. PLoS Pathog, 2019. 15(1): p. e1007530.

Garcia-Serradilla, M., C. Risco, and B. Pacheco, Drug repurposing ƒor new, eƒƒicient, broad spectrum antivirals. Virus Res, 2019. 264: p. 22-31.

Gassen, N.C., Niemeyer, D., Muth, D., Corman, V.M., Martinelli, S., Gassen, A., Hafner, K., Papies, J., Mosbauer, K., Zellner, A., et al. (2019). SKP2 attenuates autophagy through Beclin1-ubiquitination and its inhibition reduces MERS-Coronavirus infection. Nat Commun. 10(1), 5770.

Gaunt, E.R., Cheung, W., Richards, J.E., Lever, A., and Desselberger, U. (2013). Inhibition of rotavirus replication by downregulation of fatty acid synthesis. J Gen Virol. 94(Pt 6), 1310-1317.

Gillooly, D.J., Morrow, I.C., Lindsay, M., Gould, R., Bryant, N.J., Gaullier, J.M., Parton, R.G., and Stenmark, H. (2000). Localization of phosphatidylinositol 3-phosphate in yeast and mammalian cells. EMBO J. 19(17), 4577-4588.

Gillooly, D.J., Simonsen, A., and Stenmark, H. (2001). Cellular functions of phosphatidylinositol 3-phosphate and FYVE domain proteins. Biochem J. 355(Pt 2), 249-258.

Gordon, D.E., Hiatt, J., Bouhaddou, M., Rezelj, V.V., Ulferts, S., Braberg, H., Jureka, A.S., Obernier, K., Guo, J.Z., Batra, J., et al. (2020a). Comparative host-coronavirus protein interaction networks reveal pan-viral disease mechanisms. Science. 370(6521).

Gordon, D.E., Jang, G.M., Bouhaddou, M., Xu, J., Obernier, K., White, K.M., O’Meara, M.J., Rezelj, V.V., Guo, J.Z., Swaney, D.L., et al. (2020b). A SARS-CoV-2 protein interaction map reveals targets for drug repurposing. Nature. 583(7816), 459-468.

Gordon, C.J., et al., Remdesivir is α direct-acting antiviral that inhibits RNA-dependent RNA polymerase from severe acute respiratory syndrome coronavirus 2 with high potency. J Biol Chem, 2020. 295(20): p. 6785-6797.

Guo, D., E.H. Bell, and A. Chakravarti, Lipid metabolism emerges as α promising target ƒor malignant glioma therapy. CNS Oncol, 2013. 2(3): p. 289-99.

Hagemeijer, M.C., Monastyrska, I., Griffith, J., van der Sluijs, P., Voortman, J., van Bergen en Henegouwen, P.M., Vonk, A.M., Rottier, P.J., Reggiori, F., and de Haan, C.A. (2014). Membrane rearrangements mediated by coronavirus nonstructural proteins 3 and 4. Virology. 458-459, 125-135.

Halvorson, D.L., and McCune, S.A. (1984). Inhibition of fatty acid synthesis in isolated adipocytes by 5-(tetradecyloxy)-2-furoic acid. Lipids. 19(11), 851-856.

Heaton, N.S., and Randall, G. (2011). Multifaceted roles for lipids in viral infection. Trends Microbiol. 19(7), 368-375.

Hitakarun, A., Khongwichit, S., Wikan, N., Roytrakul, S., Yoksan, S., Rajakam, S., Davidson, A.D., and Smith, D.R. (2020). Evaluation of the antiviral activity of orlistat (tetrahydrolipstatin) against dengue virus, Japanese encephalitis virus, Zika virus and chikungunya virus. Sci Rep. 10(1), 1499.

Hoffmann, H.H., Schneider, W.M., Sanchez-Rivera, F.J., Luna, J.M., Ashbrook, A.W., Soto-Feliciano, Y.M., Leal, A.A., Le Pen, J., Ricardo-Lax, I., Michailidis, E., et al. (2020). Functional interrogation of a SARS-CoV-2 host protein interactome identifies unique and shared coronavirus host factors. bioRxiv.

Holshue, M.L., DeBolt, C., Lindquist, S., Lofy, K.H., Wiesman, J., Bruce, H., Spitters, C., Ericson, K., Wilkerson, S., Tural, A., et al. (2020). First Case of 2019 Novel Coronavirus in the United States. N Engl J Med. 382(10), 929-936.

Honda, A., Harrington, E., Cornella-Taracido, I., Furet, P., Knapp, M.S., Glick, M., Triantafellow, E., Dowdle, W.E., Wiedershain, D., Maniara, W., et al. (2016). Potent, Selective, and Orally Bioavailable Inhibitors of VPS34 Provide Chemical Tools to Modulate Autophagy in Vivo. ACS Med Chem Lett. 7(1), 72-76.

Hsiau, T., Conant, D., Rossi, N., Maures, T., Waite, K., Yang, J., Joshi, S., Kelso, R., Holden, K., Enzmann, B.L., et al. (2019). Inference of CRISPR Edits from Sanger Trace Data. bioRxiv. 251082. DOI: 10.1101/251082.

Igal, R.A., Wang, P., and Coleman, R.A. (1997). Triacsin C blocks de novo synthesis of glycerolipids and cholesterol esters but not recycling of fatty acid into phospholipid: evidence for functionally separate pools of acyl-CoA. Biochem J. 324 (Pt 2), 529-534.

Jaber, N., and Zong, W.X. (2013). Class IIIPI3K Vps34: essential roles in autophagy, endocytosis, and heart and liver function. Ann NY Acad Sci. 1280, 48-51.

Jureka, A.S., Silvas, J.A., and Basler, C.F. (2020). Propagation, Inactivation, and Safety Testing of SARS-CoV-2. Viruses. 12(6).

Kim, Y., et al., Novel triacsin C analogs as potential antivirals against rotavirus inƒections. Eur J Med Chem, 2012. 50: p. 311-8.

Knoops, K., Kikkert, M., Worm, S.H., Zevenhoven-Dobbe, J.C., van der Meer, Y., Koster, A.J., Mommaas, A.M., and Snijder, E.J. (2008). SARS-coronavirus replication is supported by a reticulovesicular network of modified endoplasmic reticulum. PLoS Biol. 6(9), e226.

Kridel, S.J., Axelrod, F., Rozenkrantz, N., and Smith, J.W. (2004). Orlistat is a novel inhibitor of fatty acid synthase with antitumor activity. Cancer Res. 64(6), 2070-2075.

Kuhajda, F.P., Pizer, E.S., Li, J.N., Mani, N.S., Frehywot, G.L., and Townsend, C.A. (2000). Synthesis and antitumor activity of an inhibitor of fatty acid synthase. Proc Natl Acad Sci U S A. 97(7), 3450-3454.

Kutateladze, T.G. (2007). Mechanistic similarities in docking of the FYVE and PX domains to phosphatidylinositol 3-phosphate containing membranes. Prog Lipid Res. 46(6), 315-327.

Lamb, C.A., T. Yoshimori, and S.A. Tooze, The autophagosome: origins unknown, biogenesis complex. Nat Rev Mol Cell Biol, 2013. 14(12): p. 759-74.

Lever, A., and Desselberger, U. (2016). Rotavirus replication and the role of cellular lipid droplets: New therapeutic targets? J Formos Med Assoc. 115(6), 389-394.

Li, G., and De Clercq, E. (2020). Therapeutic options for the 2019 novel coronavirus (2019-nCoV). Nat Rev Drug Discov. 19(3), 149-150.

Liefhebber, J.M., et al., Modulation oƒ triglyceride and cholesterol ester synthesis impairs assembly oƒ inƒectious hepatitis C virus. J Biol Chem, 2014. 289(31): p. 21276-88.

Lundstrom, K. (2020). Coronavirus Pandemic-Therapy and Vaccines. Biomedicines. 8(5).

Ma, Y., Wang, W., Devarakonda, T., Zhou, H., Wang, X.Y., Salloum, F.N., Spiegel, S., and Fang, X. (2020). Functional analysis of molecular and pharmacological modulators of mitochondrial fatty acid oxidation. Sci Rep. 10(1), 1450.

Maier, H.J. and P. Britton, Involvement oƒ autophagy in coronavirus replication. Viruses, 2012. 4(12): p. 3440-51.

Majeau, N., Fromentin, R., Savard, C., Duval, M., Tremblay, M.J., and Leclerc, D. (2009). Palmitoylation of hepatitis C virus core protein is important for virion production. J Biol Chem. 284(49), 33915-33925.

Martin-Acebes, M.A., N. Jiménez de Oya, and J.C. Saiz, Lipid Metabolism as α Source oƒ Druggable Targets ƒor Antiviral Discovery against Zika and Other Flaviviruses. Pharmaceuticals (Basel), 2019. 12(2).

McBride, C.E., and Machamer, C.E. (2010). Palmitoylation of SARS-CoV S protein is necessary for partitioning into detergent-resistant membranes and cell-cell fusion but not interaction with M protein. Virology. 405(1), 139-148.

Merino-Ramos, T., Vazquez-Calvo, A., Casas, J., Sobrino, F., Saiz, J.C., and Martin-Acebes, M.A. (2016). Modification of the Host Cell Lipid Metabolism Induced by Hypolipidemic Drugs Targeting the Acetyl Coenzyme A Carboxylase Impairs West Nile Virus Replication. Antimicrob Agents Chemother. 60(1), 307-315.

Nasheri, N., et al., Modulation of fatty acid synthase enzyme activity and expression during hepatitis C virus replication. Chem Biol, 2013. 20(4): p. 570-82.

Ohashi, Y., Tremel, S., Masson, G.R., McGinney, L., Boulanger, J., Rostislavleva, K., Johnson, C.M., Niewczas, I., Clark, J., and Williams, R.L. (2020). Membrane characteristics tune activities of endosomal and autophagic human VPS34 complexes. Elife. 9.

Oudshoorn, D., Rijs, K., Limpens, R., Groen, K., Koster, A.J., Snijder, E.J., Kikkert, M., and Barcena, M. (2017). Expression and Cleavage of Middle East Respiratory Syndrome Coronavirus nsp3-4 Polyprotein Induce the Formation of Double-Membrane Vesicles That Mimic Those Associated with Coronaviral RNA Replication. mBio. 8(6).

Pasquier, B. (2015). SAR405, a PIK3C3/Vps34 inhibitor that prevents autophagy and synergizes with MTOR inhibition in tumor cells. Autophagy. 11(4), 725-726.

Petit, C.M., Chouljenko, V.N., Iyer, A., Colgrove, R., Farzan, M., Knipe, D.M., and Kousoulas, K.G. (2007). Palmitoylation of the cysteine-rich endodomain of the SARS-coronavirus spike glycoprotein is important for spike-mediated cell fusion. Virology. 360(2), 264-274.

Pizzorno, A., et al., Drug Repurposing Approaches ƒor the Treatment oƒ Influenza Viral Inƒection: Reviving Old Drugs to Fight Against α Long-Lived Enemy. Front Immunol, 2019. 10: p. 531.

Prentice, E., et al., Coronavirus replication complex ƒormation utilizes components oƒ cellular autophagy. J Biol Chem, 2004. 279(11): p. 10136-41.

Reggiori, F., C.A. de Haan, and M. Molinari, Unconventional use oƒ LC3 by coronaviruses through the alleged subversion oƒ the ERAD tuning pathway. Viruses, 2011. 3(9): p. 1610-23.

Reggiori, F., et al., Coronaviruses Hijack the LC3-I-positive EDEMosomes, ER-derived vesicles exporting short-lived ERAD regulators, ƒor replication. Cell Host Microbe, 2010. 7(6): p. 500-8.

Ronan, B., Flamand, O., Vescovi, L., Dureuil, C., Durand, L., Fassy, F., Bachelot, M.F., Lamberton, A., Mathieu, M., Bertrand, T., et al. (2014). A highly potent and selective Vps34 inhibitor alters vesicle trafficking and autophagy. Nat Chem Biol. 10(12), 1013-1019.

Ruzica Bago 1, Nazma Malik 1, Michael J Munson 1, Alan R Prescott 2, Paul Davies 1, Eeva Sommer 1, Natalia Shpiro 1, Richard Ward 3, Darren Cross 3, Ian G Ganley 1, Dario R Alessi

Saini, K.S., Lanza, C., Romano, M., de Azambuja, E., Cortes, J., de Las Heras, B., de Castro, J., Lamba Saini, M., Loibl, S., Curigliano, G., et al. (2020). Repurposing anticancer drugs for COVID-19-induced inflammation, immune dysfunction, and coagulopathy. Br J Cancer. 123(5), 694-697.

Schneider, W.M., Luna, J.M., Hoffmann, H.H., Sanchez-Rivera, F.J., Leal, A.A., Ashbrook, A.W., Le Pen, J., Ricardo-Lax, I., Michailidis, E., Peace, A., et al. (2021). Genome-Scale Identification of SARS-CoV-2 and Pan-coronavirus Host Factor Networks. Cell. 184(1), 120-132 e114.

Schütter, M., et al., Local Fatty Acid Channeling into Phospholipid Synthesis Drives Phagophore Expansion during Autophagy. Cell, 2020. 180(1): p. 135-149.e14.

Silvas, J.A., et al., Extracellular Vesicles Mediate Receptor-Independent Transmission of Novel Tick-Borne Bunyavirus. J Virol, 2016. 90(2): p. 873-86.

Sims, A.C., et al., SARS-CoV replication and pathogenesis in an in vitro model oƒ the human conducting airway epithelium. Virus Res, 2008. 133(1): p. 33-44.

Sims, A.C., et al., Severe acute respiratory syndrome coronavirus inƒection oƒ human ciliated airway epithelia: role oƒ ciliated cells in viral spread in the conducting airways oƒ the lungs. J Virol, 2005. 79(24): p. 15511-24.

Snijder, E.J., Limpens, R., de Wilde, A.H., de Jong, A.W.M., Zevenhoven-Dobbe, J.C., Maier, H.J., Faas, F., Koster, A.J., and Barcena, M. (2020). A unifying structural and functional model of the coronavirus replication organelle: Tracking down RNA synthesis. PLoS Biol. 18(6), e3000715.

Stjepanovic, G., et al., Unveiling the role oƒ VPS34 kinase domain dynamics in regulation oƒ the autophagic PI3K complex. Mol Cell Oncol, 2017. 4(6): p. e1367873.

Stjepanovic, G., et al., Vps34 Kinase Domain Dynamics Regulate the Autophagic PI 3-Kinase Complex. Mol Cell, 2017. 67(3): p. 528-534.e3.

Su, W.C., et al., Rab5 and class IIIphosphoinositide 3-kinase Vps34 are involved in hepatitis C virus NS4B-inducedautophagy. J Virol, 2011. 85(20): p. 10561-71.

Thorp, E.B., Boscarino, J.A., Logan, H.L., Goletz, J.T., and Gallagher, T.M. (2006). Palmitoylations on murine coronavirus spike proteins are essential for virion assembly and infectivity. J Virol. 80(3), 1280-1289.

Tongluan, N., et al., Involvement of fatty acid synthase in dengue virus infection. Virol J, 2017. 14(1): p. 28.

Tseng, Y.T., Wang, S.M., Huang, K.J., and Wang, C.T. (2014). SARS-CoV envelope protein palmitoylation or nucleocapid association is not required for promoting virus-like particle production. J Biomed Sci. 21, 34.

V′Kovski, P., Kratzel, A., Steiner, S., Stalder, H., and Thiel, V. (2021). Coronavirus biology and replication: implications for SARS-CoV-2. Nat Rev Microbiol. 19(3), 155-170.

Wakil, S.J., and Abu-Elheiga, L.A. (2009). Fatty acid metabolism: target for metabolic syndrome. J Lipid Res. 50 Suppl, S138-143.

Wang, L., et al., Review oƒ the 2019 novel coronavirus (SARS-CoV-2) based on current evidence. Int J Antimicrob Agents, 2020. 55(6): p. 105948.

Wang, R., Simoneau, C.R., Kulsuptrakul, J., Bouhaddou, M., Travisano, K.A., Hayashi, J.M., Carlson-Stevermer, J., Zengel, J.R., Richards, C.M., Fozouni, P., et al. (2021). Genetic Screens Identify Host Factors for SARS-CoV-2 and Common Cold Coronaviruses. Cell. 184(1), 106-119 e114.

Weber, F., Wagner, V., Rasmussen, S.B., Hartmann, R., and Paludan, S.R. (2006). Doublestranded RNA is produced by positive-strand RNA viruses and DNA viruses but not in detectable amounts by negative-strand RNA viruses. J Virol. 80(10), 5059-5064. Wu, J., B. Wu, and T. Lai, Compassionate Use oƒ Remdesivir in Covid-19. N Engl J Med, 2020. 382.

Wu, X., et al., Roles oƒ viroplasm-like structures formed by nonstructural protein NSs in infection with severe fever with thrombocytopenia syndrome virus. FASEB J, 2014. 28(6): p. 2504-16.

Yu, G.Y., Lee, K.J., Gao, L., and Lai, M.M. (2006). Palmitoylation and polymerization of hepatitis C virus NS4B protein. J Virol. 80(12), 6013-6023.

Yuen, C.K., Wong, W.M., Mak, L.F., Wang, X., Chu, H., Yuen, K.Y., and Kok, K.H. (2021). Suppression of SARS-CoV-2 infection in ex-vivo human lung tissues by targeting class III phosphoinositide 3-kinase. J Med Virol. 93(4), 2076-2083.

Zang, R., Case, J.B., Yutuc, E., Ma, X., Shen, S., Gomez Castro, M.F., Liu, Z., Zeng, Q., Zhao, H., Son, J., et al. (2020). Cholesterol 25-hydroxylase suppresses SARS-CoV-2 replication by blocking membrane fusion. Proc Natl Acad Sci U S A. 117(50), 32105-32113.

Zhao, G., Souers, A.J., Voorbach, M., Falls, H.D., Droz, B., Brodjian, S., Lau, Y.Y., Iyengar, R.R., Gao, J., Judd, A.S., et al. (2008). Validation of diacyl glycerolacyltransferase I as a novel target for the treatment of obesity and dyslipidemia using a potent and selective small molecule inhibitor. J Med Chem. 51(3), 380-383.

Zhao, Z., Thackray, L.B., Miller, B.C., Lynn, T.M., Becker, M.M., Ward, E., Mizushima, N.N., Denison, M.R., and Virgin, H.W.t. (2007). Coronavirus replication does not require the autophagy gene ATG5. Autophagy. 3(6), 581-585.

Zhu, N., Zhang, D., Wang, W., Li, X., Yang, B., Song, J., Zhao, X., Huang, B., Shi, W., Lu, R., et al. (2020). A Novel Coronavirus from Patients with Pneumonia in China, 2019. N Engl J Med. 382(8), 727-733.

What is claimed is:

1. A method of treating a viral infection in a subject comprising administering to the subject an inhibitor of PI3K, an inhibitor of lipases and/or fatty acid synthase, an inhibitor of fatty acyl-CoA-synthetases (ACS), an inhibitor of diacylglycerol acyltransferase 1 (DGAT1), a palmitoyl acyltransferases (PAT) inhibitor, and/or an inhibitor of fatty acyl-CoA-carboxylases (ACC).

2. The method of treating a viral infection of claim 1, wherein the PI3K inhibitor is a VPS34 inhibitor.

3. (canceled)

4. The method of treating a viral infection of claim 1, wherein the VPS34 inhibitor is a small molecule inhibitor comprising VPS34 IN1, PIK-III, SB02024, compound 19, or SAR405 HY12481.

5. (canceled)

6. The method of treating a viral infection of claim 1, wherein the lipase and/or fatty acid synthase inhibitor is a small molecule comprising Orlistat or C75; and/or wherein the fatty acyl-CoA-carboxylase inhibitor is a small molecule comprising 5-(Tetradecyloxy)-2-furoic acid (TOFA) and/or wherein the fatty acyl-CoA-synthetases inhibitor is a small molecule comprising Triacsin C.

7-8. (canceled)

9. The method of treating a viral infection of claim 1, wherein inhibitor of diacylglycerol acyltransferase 1 (DGAT1) comprises A922500.

10. The method of treating a viral infection of claim 1, wherein inhibitor of palmitoyl acyltransferases (PAT) comprise 2-bromopalmitate.

11. The method of treating a viral infection of claim 1, wherein the virus comprises a coronavirus selected from the group consisting of avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2), and middle east respiratory syndrome (MERS) coronavirus (CoV) (MERS-CoV); or wherein the virus comprises hepatitis C virus; or wherein wherein the virus comprises a tombusvirus selected from the group consisting of artichoke mottled crinkle virus (AMCV), carnation Italian ringspot virus (CIRV), cucumber Bulgarian virus (CBLV), cucumber necrosis virus (CuNV), cumbidium ringspot virus (CymRSV), eggplant mottled crinkle virus (EMCV), grapevine Algerian latent virus (GALV), tomato bushy stunt virus (TBSV), Petunia asteroid mosaic virus (PetAMV), Pelargonium leaf curl virus (PLCV), Moroccan pepper virus (MPV), Limonium flower distortion virus (LFDV), Havel River virus (HRV), Lato river virus (LRV), Sitke waterborne virus (SWBV), and Neckar River virus (NRV); or wherein the virus comprises a rotasvirus selected from the group consisting of rotavirus A, rotavirus B, rotavirus C, rotavirus D, rotavirus E, rotavirus F, rotavirus G, rotavirus H, rotavirus I, and rotavirus J.

12-14. (canceled)

15. A method of screening for a therapeutic agent for the treatment, inhibition, reduction, amelioration, and/or prevention of a viral infection the method comprising contacting a monolayer of cells with the therapeutic agent; infecting the monolayer of cells with the virus creating an infected cell monolayer; incubating the infected cell monolayer; and measuring impedance across the monolayer; wherein a decrease in the impedance indicates viral growth, and wherein the impedance at which 50% of the monolayer cells are dead is the 50% inhibitor concentration (IC50), thereby indicating that the therapeutic agent treats, inhibits, reduces ameliorates, and/or prevents viral infection.

16. The method of screening a therapeutic agent of claim 15, further comprising establishing an incubation time to result in 50% death of infected, but untreated cells for a given multiplicity of infection.

17. The method of screening a therapeutic agent of claim 15, further comprising comparing the IC50 with uninfected cell controls and/or infected and untreated cell controls.

18. The method of screening a therapeutic agent of claim 15, wherein the therapeutic agent is added to the monolayer prior to infection.

19. (canceled)

20. The method of screening a therapeutic agent of claim 15, wherein the therapeutic agent is added to the monolayer after infection.

21-25. (canceled)

26. A method of inhibiting viral replication comprising contacting a virus with an inhibitor of fatty acid synthesis.

27. The method of inhibiting viral replication of claim 26, wherein the inhibitor of fatty acid synthesis comprises a protein palmitoylation inhibitor, an inhibitor of fatty acyl-CoA-synthetases (ACS), an inhibitor of diacylglycerol acyltransferase 1 (DGAT1), and/or an inhibitor of fatty acyl-CoA-carboxylases (ACC).

28. The method of inhibiting viral replication of claim 27, wherein the protein palmitoylation inhibitor comprises a palmitoyl acyltransferases (PAT) inhibitor.

29. The method of inhibiting viral replication of claim 28, wherein the PAT inhibitor comprises 2- bromopalmitate.

30. The method of inhibiting viral replication of claim 27, wherein the lipase and/or fatty acid synthase inhibitor is a small molecule comprising Orlistat or C75; and/or wherein the fatty acyl-CoA-carboxylase inhibitor is a small molecule comprising 5-(Tetradecyloxy)-2-furoic acid (TOFA) and/or wherein the fatty acyl-CoA-synthetases inhibitor is a small molecule comprising Triacsin C.

31-32. (canceled)

33. The method of inhibiting viral replication of claim 27, wherein inhibitor of diacylglycerol acyltransferase 1 (DGAT1) comprises A922500.

34. The method of inhibiting viral replication of claim 26, wherein the virus is a coronavirus selected from the group consisting of avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2), and middle east respiratory syndrome (MERS) coronavirus (CoV) (MERS-CoV); or wherein the virus comprises hepatitis C virus; or wherein wherein the virus comprises a tombusvirus selected from the group consisting of artichoke mottled crinkle virus (AMCV), carnation Italian ringspot virus (CIRV), cucumber Bulgarian virus (CBLV), cucumber necrosis virus (CuNV), cumbidium ringspot virus (CymRSV), eggplant mottled crinkle virus (EMCV), grapevine Algerian latent virus (GALV), tomato bushy stunt virus (TBSV), Petunia asteroid mosaic virus (PetAMV), Pelargonium leaf curl virus (PLCV), Moroccan pepper virus (MPV), Limonium flower distortion virus (LFDV), Havel River virus (HRV), Lato river virus (LRV), Sitke waterborne virus (SWBV), and Neckar River virus (NRV); or wherein the virus comprises a rotasvirus selected from the group consisting of rotavirus A, rotavirus B, rotavirus C, rotavirus D, rotavirus E, rotavirus F, rotavirus G, rotavirus H, rotavirus I, and rotavirus J.

35-37. (canceled)
